(19) United States
(12) Patent Application Publication
(10) Pub. No.: US 2021/0340576 A1
(43) Pub. Date: Nov. 4, 2021

(54) METHOD FOR THE MONITORING OF MODIFIED NUCLEASES INDUCED-GENE EDITING EVENTS BY MOLECULAR COMBING

(71) Applicant: GENOMIC VISION SA, Bagneux (FR)

(72) Inventors: Sebastien BARRADEAU, Paris (FR); Aaron BENSIMON, Antony (FR); Laurent CAVAREC, Vincennes (FR)

(73) Assignee: GENOMIC VISION SA, Bagneux (FR)

(21) Appl. No.: 17/366,643

(22) Filed: Jul. 2, 2021

Related U.S. Application Data
(63) Continuation of application No. 15/813,974, filed on Nov. 15, 2017.
(60) Provisional application No. 62/422,341, filed on Nov. 15, 2016.

(51) Int. Cl. C12N 15/90; C12Q 1/6841; C12Q 1/6827; C12N 15/10; C12N 15/11; C12Q 1/6816

(52) U.S. Cl. CPC C12N 15/907; C12Q 1/6841; C12Q 1/6827; C12N 15/102; C12N 2800/80; C12Q 1/6816; C12Q 2537/143; C12N 2310/20; C12N 2320/11; C12N 15/11

(57) ABSTRACT

Methods for detecting and characterizing large genomic rearrangements induced by modified nucleases at high resolution and for quantifying the frequency of the large genomic or gene rearrangements induced by modified nucleases using Molecular Combing.

### BACKGROUND OF THE INVENTION

### Field of the Invention

This invention is related to a method for detecting and characterizing large genomic rearrangements induced by modified nucleases at high resolution using Molecular Combing. This invention also relates a method using Molecular Combing to quantify the frequency of the large genomic rearrangements induced by modified nucleases.

### Description of the Related Art

Molecular Combing

Molecular combing technology has been disclosed in various patents and scientific publications, for example in U.S. Pat. No. 6,303,296, WO 9818959, WO 0073503, U.S. 2006/257910, U.S. 2004/033510, U.S. Pat. Nos. 6,130,044, 6,225,055, 6,054,327, WO 2008/028931, WO 2010/035140, and in (Michalet, Ekong et al. 1997; Herrick, Michalet et al. 2000; Herrick, Stanislawski et al. 2000; Gad, Aurias et al. 2001; Gad, Caux-Moncoutier et al. 2002; Gad, Klinger et al. 2002; Herrick, Jun et al. 2002; Pasero, Bensimon et al. 2002; Lebofsky and Bensimon 2003; Jun, Herrick et al. 2004; Caburet, Conti et al. 2005; Herrick, Conti et al. 2005; Lebofsky and Bensimon 2005; Lebofsky, Heilig et al. 2006; Patel, Arcangioli et al. 2006; Rao, Conti et al. 2007; Schurra and Bensimon 2009; Nguyen, Walrafen et al. 2011; Cheeseman, Rouleau et al. 2012; Mahiet, Ergani et al. 2012; Tessereau, Buisson et al. 2013; Cheeseman, Ropars et al. 2014; Tessereau, Lesecque et al. 2014; Vasale, Boyar et al. 2015). The techniques of these references, specifically those pertaining or relating to molecular combing, are hereby incorporated by reference to the publications cited above.

Bensimon, et al., U.S. Pat. No. 6,303,296 discloses DNA stretching procedures, Lebofsky, et al., WO 2008/028931 also discloses Molecular Combing procedures.

Stretching nucleic acid, extracted from any source (from virus, bacteria to human through plants . . . ), provides immobilized nucleic acids in linear and parallel strands and is preferably preformed with a controlled stretching factor on an appropriate surface (e.g., surface-treated glass slides). After stretching, it is possible to hybridize sequence-specific probes detectable for example by fluorescence microscopy (Lebofsky, Heilig et al. 2006). Thus, a particular sequence may be directly visualized on a single molecule level. The length of the fluorescent signals and/or their number, and their spacing on the slide provides a direct reading of the size and relative spacing of the probes.

Molecular combing is a technique enabling the direct visualization of individual nucleic acid molecules and has numerous applications for DNA structural such as physical mapping (Michalet, Ekong et al. 1997; Tessereau, Buisson et al. 2013; Cheeseman, Ropars et al. 2014) and detection of rearrangements including deletions and amplifications like in the Ca²⁺-activated neutral protease 3 gene involved in the tuberous sclerosis (Michalet, Ekong et al. 1997) and in the BRCA1 and BRCA2 genes that confer predisposition to the hereditary breast and ovarian cancer syndrome (Gad, Aurias et al. 2001; Gad, Caux-Moncoutier et al. 2002; Gad, Klinger et al. 2002; Gad, Bieche et al. 2003; Cheeseman, Rouleau et al. 2012). WO2014140788 A1 and WO2014140789 A1 disclose a method for detecting the amplifications of sequences in the BRCA1 locus and for the detection of breakpoints in rearranged genomic sequences, respectively. WO2013064895 A1 discloses for detecting genomic rearrangements in BRCA1 and BRCA2 genes at high resolution using Molecular Combing and for determining a predisposition to a disease or disorder associated with these rearrangements including predisposition to ovarian cancer or breast cancer.

Molecular Combing has also been successfully to determine the number of gene copies, for example in the trisomy 21 (Herrick, Michalet et al. 2000), to elucidate the organization of repeats regions such as human ribosomal DNA (Caburet, Conti et al. 2005), D4Z4 (Nguyen, Walrafen et al. 2011) and RNU2 arrays (Tessereau, Buisson et al. 2013; Tessereau, Lesecque et al. 2014; Tessereau, Leone et al. 2015) and to detect integration of exogenous DNA such as viral integration (Herrick, Conti et al. 2005; Conti, Herrick et al. 2007). WO 2010/035140 A1 discloses a method for analysis of D4Z4 tandem repeat arrays on human chromosomes 4 and 10 based on stretching of nucleic acid and on molecular combing.

Molecular Combing also applied to functional studies for the characterization of DNA replication (Herrick, Stanislawski et al. 2000; Herrick, Jun et al. 2002; Lebofsky and Bensimon 2003; Lebofsky and Bensimon 2005; Lebofsky, Heilig et al. 2006; Bailis, Luche et al. 2008; Daboussi, Courbet et al. 2008; Dorn, Chastain et al. 2009; Schurra and Bensimon 2009), DNA/protein interaction (Herrick and Bensimon 1999) and transcription (Gueroui, Place et al. 2002).

The patents referenced below describe various molecular combing procedures and individual steps useful in configuring a molecular combing procedure tailored to a particular purpose. Based on the present disclosure, those skilled in the art may adapt these procedures or their individual steps to detect, quantify or otherwise characterize genome or gene editing events performed by CRISPR-Cas9, other CRISPR-based or other genome or gene editing procedures.

One example of molecular combing from U.S. Pat. No. 6,303,296 comprises aligning a nucleic acid on a surface S of a support, wherein the process comprises: (a) providing a support having a surface S; (b) contacting the surface S with the nucleic acid; (c) anchoring the nucleic acid to the surface S; (d) contacting the surface S with a first solvent A; (e) contacting the first solvent A with a medium B to form an A/B interface, wherein said medium B is a gas or a second solvent; (f) forming a triple line S/A/B (meniscus) resulting from the contact between the first solvent A, the surface S, and the medium B; and (g) moving the meniscus to align the nucleic acid on the surface.

Another example, based on the disclosure of U.S. Pat. No. 7,985,542 comprises a method of detecting the presence of at least one domain of interest on a macromolecule to test that comprises: a) determining at least three target regions on the domain of interest, b) obtaining a corresponding labelled set of at least three probes each probe targeting one of said target region, the position of the probes one compared to the others being chosen and forming a sequence of at least two codes chosen between a group of at least two different codes, said sequence of codes being specific of the domain and being a specific signature of said domain of interest on the macromolecule to test; c) spreading the macromolecule and binding the probes to the macromolecule, wherein the spreading step occurs before or after the binding step, d) reading signals given by each of the labelled probes, each signal being associated with the label of said one probe, e) transcribing said signals in a sequence of codes established from the gap size between consecutive probes, f) detecting the sequence of codes of a domain of interest said sequence indicating the presence of said domain of interest on the macromolecule to test, and conversely the absence of detection of sequence of codes or part of sequence of codes of a domain of interest indicating the absence of said domain or part of said domain of interest on the macromolecule to test.

A third example of molecular combing based on the disclosure of U.S. Pat. No. 7,732,143 comprises a method of identifying a genetic abnormality comprising a break in a genome, wherein the method comprises: (a) providing a surface on which genomic DNA comprising a plurality of clones has been aligned using a molecular combing technique; (b) contacting the genomic DNA with at least one probe that is specific for a genomic sequence for which the genetic abnormality is sought; (c) detecting a hybridization signal between the at least one probe and the genomic DNA; (d) identifying the presence of the break in the genome directly or by comparing the length of the sequences detected by the hybridization signal to the length of sequences detected by a hybridization signal obtained using a control genome that does not contain the break and the at least one probe of part (b), and (e) determining the number of clones having a defined probe length, wherein the determined numbers of clones and the lengths of the sequences detected by the hybridization signals are converted into a graph.

None of these patents referenced above contemplated using molecular combing in combination with CRISPR-Cas9 like genomic or gene editing or the advantages attained by this combination including the avoidance of bias and the improved efficiency provided by a single assay as disclosed herein.

Repair of DNA Double Strand Breaks

Double strand breaks (DSB) in DNA are common events in eukaryotic cells that may induce deleterious damages and subsequently to genome instability and/or cell death. These events are typically repaired through either non-homologous end-joining (NHEJ) or homologous recombination (HR) pathways (Takata, Sasaki et al. 1998).

Genome editing by NHEJ generally results in small deletions and/or insertions (indels) at the site of the break. NHEJ is an error prone mechanism that functions to repair DSBs without a template through direct relegation of the cleaved ends. This can create a frameshift mutation that may knockout gene function by a combination of two mechanisms: premature truncation of the encoded protein and non-sense-mediated decay of the mRNA transcript. NHEJ can occur during any phase of the cell cycle. In higher eukaryotes, NHEJ, rather than HR, is the dominant DSB repair system (Bibikova, Golic et al. 2002; Puchta 2005; Lieber 2010; Lieber and Wilson 2010).

HR relies on strand invasion of the broken end into a homologous sequence and subsequent repair of the break in a template-dependent manner (Szostak, Orr-Weaver et al. 1983). HR can be mediated by four different conservative and non-conservative mechanisms:

Gene Conversion (GC).

GC is basically initiated by the DSB formation at the recombination-recipient sites. The DSB ends are processed to have single stranded DNA tails, one of which eventually invades into the duplex of unbroken DNA. The invaded single strand DNA tail then forms a heteroduplex with the homologous DNA stretch in the unbroken template strand. The free DNA end of this heteroduplex primes a repair DNA synthesis. After a strand extension, the newly synthesized strand dissociates form the unbroken template DNA and anneals with the original broken DNA. Finally, the single strand DNA gap is filled followed by a ligation of DNA nicks. In this process, the DNA sequence on the unbroken DNA strand is converted to the broken strand, thereby accompanying a unidirectional transfer of genetic information (Paques and Haber 1999; Allers and Lichten 2001; Allers and Lichten 2001).

Non-Allelic Homologous Recombination (NAHR).

Indeed, HR can also occur ectopically between highly similar duplicated sequences or paralogous genomic segments, such as segmental duplications, through NAHR mechanism. NAHR can occur between directly oriented duplicated sequences on the same chromosome giving rise to a chromosomal deletion, and, if it occurs in an intermolecular fashion, it can generate a reciprocal duplication on the other chromosome. When NAHR takes place between duplicated sequences in an inverted orientation, it leads to inversions. NAHR is a mechanism leading to genomic variations and genomic disorders.

Break-Induced Replication (BIR).

BIR pathway is employed to repair a DSB when homology is restricted to one end. In that case, recombination is used to establish a unidirectional replication fork that can copy the donor template to the end of the chromosome (McEachern and Haber 2006; Llorente, Smith et al. 2008). BIR mechanism is responsible of some segmental duplications (Payen, Koszul et al. 2008), deletions, nonreciprocal translocations, and complex rearrangements seen in a number of human diseases and cancers (Hastings, Lupski et al. 2009).

Single Strand Annealing (SSA).

SSA is restricted to repair of DNA breaks that are flanked by direct repeats that can be as short as 30 nucleotides (Sugawara, Ira et al. 2000; Villarreal, Lee et al. 2012). Resection exposes the complementary strands of homologous sequences, which recombine resulting in a deletion containing a single copy of the repeated sequences through removal of the non-homologous single-stranded tails by the Rad1-Rad10 endonuclease complex (XPF-ERCC1 in mammals). SSA is therefore considered to be highly mutagenic.

When an exogenous DNA donor that has homologous sequences flanking the DSB is introduced along with the modified nuclease, the cell's machinery will use the supplied donor sequence as template for repair, thereby creating precise nucleotide change at or near the DSB site (Rouet, Smih et al. 1994). The length of the homologous region may vary between 70 to several hundred base pairs according to the nature of the donor DNA (single-stranded oligonucleotides or plasmids) (Yang, Guell et al. 2013; Hendel, Kildebeck et al. 2014). The donor DNA can be used to introduce either precise nucleotide substitutions or deletions, endogenous gene labelling, and targeted gene addition (McMahon, Randar et al. 2012). It has been shown that efficiency of gene targeting through HR in mammalian cells is stimulated by several orders of magnitude by introduction of DSB at the target site (Rouet, Smih et al. 1994; Choulika, Perrin et al. 1995; Smih, Rouet et al. 1995).

Genome Editing

Genome editing with engineered nucleases is a technology that allows targeted modifications of any genomic DNA sequences (Baker 2012). This technology relies on the activation of the endogenous cellular repair machinery by DNA DSB through HR or NHEJ mechanisms as described above.

Four major types of nucleases exist to create targeted DNA DSB at specific site: zinc-finger nucleases (ZFNs), transcription activator-like effector-nuclease (TALENs), meganucleases and the CRISPR/Cas9 system (For review, (Maeder and Gersbach 2016; Merkert and Martin 2016).

Zinc Finger Nucleases

The zinc finger nuclease (ZFN)-based technology is based on the fact that the DNA-binding domain and the cleavage domain of the FokI restriction endonuclease function independently of each other (Li, Wu et al. 1992). Thus, chimeric nucleases with novel binding specificities can be produced by replacing the FokI DNA-binding domain with a zinc finger domain (Kim and Chandrasegaran 1994; Kim, Cha et al. 1996). Since ZFN-induced DSBs could be used to modify the genome through either NHEJ or HR (Bibikova, Carroll et al. 2001; Porteus and Baltimore 2003), this technology can be used to modify genes in both human somatic and pluripotent stem cell (For review: (Jo, Kim et al. 2015; Vasileva, Shuvalov et al. 2015).

TALENs

The discovery of a simple one-to-one code dictating the DNA-binding specificity of TALE proteins from the plant pathogen Xanthomonas again raised the exciting possibility for modular design of novel DNA-binding proteins (Boch, Scholze et al. 2009; Moscou and Bogdanove 2009). The DNA binding domain contains a repeated highly conserved 33-34 amino acid sequence with divergent 12^{th} and 13^{th} amino acids. These two positions, referred to as the Repeat Variable Diresidue (RVD), are highly variable and show a strong correlation with specific nucleotide recognition. This relationship between amino acid sequence and DNA recognition allowed the selection of a combination of repeat segments containing the appropriate RVDs to target specific regions. This discovery of TALEs as a programmable DNA-binding domain was rapidly followed by the engineering of TALENs. Like ZFNs, TALEs were fused to the catalytic domain of the FokI endonuclease and shown to function as dimers to cleave their intended DNA target site (Christian, Cermak et al. 2010; Miller, Tan et al. 2011). Also similar to ZFNs, TALENs have been shown to efficiently induce both NHEJ and HR in human both somatic and pluripotent stem cells (For review, (Vasileva, Shuvalov et al. 2015; Merkert and Martin 2016).

Meganucleases

Meganuclease technology involves re-engineering the DNA-binding specificity of naturally occurring homing endonucleases characterized by a large recognition site (double-stranded DNA sequences of 12 to 40 base pairs). There are currently six known families of meganucleases with conserved structural motifs: LAGLIDADG (SEQ. ID NO: 1), HNH, His-Cys box, GYI-YIG, PD-(D/E)xk and Vsr-like families (Belfort and Roberts 1997, incorporated by reference). The largest class of homing endonucleases is the LAGLIDADG (SEQ. ID NO: 1) family, which includes the well-characterized and commonly used I-CreI and I-SceI enzymes (Cohen-Tannoudji, Robine et al. 1998; Chevalier and Stoddard 2001). Through a combination of rational design and selection, these homing endonucleases can be re-engineered to target novel sequences (Arnould, Perez et al. 2007; Grizot, Smith et al. 2009) and showed promise for the use of meganucleases in genome editing (Redondo, Prieto et al. 2008; Dupuy, Valton et al. 2013).

CRISPR/Cas9 System

CRISPR-Cas RNA-guided nucleases are derived from an adaptive immune system that evolved in bacteria to defend against invading plasmids and viruses (Barrangou, Fremaux et al. 2007). Six major types of CRISPR system have been identified from different organisms (types I-VI) with various subtypes in each major type (Chylinski, Makarova et al. 2014; Makarova, Wolf et al. 2015). Within the type II CRISPR system, several species of Cas9 have been characterized from Streptococcus (S.) pyogenes, S. thermophilus, Neisseria meningitidis, S. aureus and Francisella novicida, so far (Gasiunas, Barrangou et al. 2012; Jinek, Chylinski et al. 2012; Mali, Aach et al. 2013; Sampson, Saroj et al. 2013; Zhang, Heidrich et al. 2013; Ran, Cong et al. 2015; Hirano, Gootenberg et al. 2016).

Three components are required for the CRISPR nuclease system to dictate specificity of DNA cleavage through Watson-Crick base pairing between nucleic acids: the CRISPR-associated (Cas) 9 protein, the mature CRISPR RNAs (crRNA) and a trans-activating crRNAs (tracrRNA) (Deltcheva, Chylinski et al. 2011). It has been showed that this system could be reduced to two components by fusion of the crRNA and tracrRNA into a single guide RNA (gRNA) (Jinek, Chylinski et al. 2012). To search for a DNA target, Cas9 nuclease only requires a 20-nucleotide sequence on the gRNA that base pairs with the target DNA and a DNA protospacer adjacent motif (PAM) adjacent to the complementary sequence (Marraffini and Sontheimer 2010; Jinek, Chylinski et al. 2012). Furthermore, re-targeting of the Cas9/gRNA complex to new sites could be accomplished by altering the sequence of a short portion of the gRNA.

While most of the Cas9 have similar RNA-guided DNA binding DNA mechanism, they often have distinct PAM recognition motif(s) expanding the targetable genome sequence for gene editing and genome manipulation. Furthermore, some types of CRISPR system may exhibit different mechanisms. For example, the type III-B CRISPR system from Pyrococcus furiosus uses a Cas complex for RNA-directed RNA cleavage that allows targeting and modulation of RNAs in cells (Hale, Zhao et al. 2009; Hale, Majumdar et al. 2012). Recently, it has been shown that the protein Cpf1 (type V) isolated from Prevotela and Francisella uses a short crRNA without a tracrRNA for RNA-guided DNA cleavage and Cpf1-mediated genome targeting is effective and specific, comparable with the S. pyogenes Cas9 (Zetsche, Gootenberg et al. 2015; Dong, Ren et al. 2016; Fonfara, Richter et al. 2016; Yamano, Nishimasu et al. 2016). Finally, the type VI-A CRISPR effector C2c2 from Leptotrichia shahii is a RNA-guided RNase that can be programmed to knock down specific mRNAs in bacterium (Abudayyeh, Gootenberg et al. 2016). This diversity in natural CRISPR/Cas Systems may provide a functionally diverse set of editing tools.

Variants of the Cas9 system have also been developed. For example, a mutant form, known as Cas9D10A, with only nickase activity that can cleave only one strand and, subsequently only activate HR pathway when provided with a homologous repair template (Cong, Ran et al. 2013). Cas9D10A can even enhance specificity of gene editing by using a pair of Cas9D10A that target each strand of DNA at adjacent sites (Ran, Hsu et al. 2013). A nuclease deficient Cas9 (dCas9) that still has the capability to bind DNA is used to sequence-specifically target any region of the genome without cleavage. Instead, by fusing with various effector domain, dCas9 can be used as a gene silencing or activation tool (Maeder, Linder et al. 2013) or as a visualization tool when fused with fluorescent protein (Chen and Huang 2014).

In contrast to ZNFs, TALENs and meganucleases that described above, the CRISPR/Cas system does not require the engineering of novel proteins for each DNA target site. New sites can be targeted, simply by altering the short region of the gRNA that dictates specificity. Additionally, because the Cas9 protein is not directly coupled to the gRNA, this system is highly amenable to multiplexing through the concurrent use of multiple gRNAs to induce DSBs at several loci. Thereafter, numerous works demonstrated that the CRISPR/Cas9 system, mainly derived from the type II CRISPR system isolated from S. pyogenes, could be engineered for efficient genetic modification in mammalian cells (Cho, Kim et al. 2013; Cong, Ran et al. 2013; Mali, Yang et al. 2013) and to generate transgenic or knock-out animal models, from worm to monkey. The two patents mentioned below describe CRISPR-Cas9 or similar genome or gene editing procedures as well as individual steps useful in these procedures. Based on the present disclosure, those skilled in the art may adapt these genome or gene editing procedures or their individual steps to modify or edit a target polynucleotide.

A representative, but not limited, CRISPR system includes that disclosed by Zhang, U.S. Pat. No. 8,795,965 comprising a method of altering expression of at least one gene product comprising introducing into a eukaryotic cell containing and expressing a DNA molecule having a target sequence and encoding the gene product an engineered, non-naturally occurring Clustered Regularly Interspaced Short Palindromic Repeats (CRISPR)-CRISPR associated (Cas) system comprising one or more vectors comprising: a) a first regulatory element operable in a eukaryotic cell operably linked to at least one nucleotide sequence encoding a CRISPR-Cas system guide RNA that hybridizes with the target sequence, and b) a second regulatory element operable in a eukaryotic cell operably linked to a nucleotide sequence encoding a Type-II Cas9 protein, wherein components (a) and (b) are located on same or different vectors of the system, wherein the guide RNA is comprised of a chimeric RNA and includes a guide sequence and a trans-activating cr (tracr) sequence, whereby the guide RNA targets the target sequence and the Cas9 protein cleaves the DNA molecule, whereby expression of the at least one gene product is altered; and, wherein the Cas9 protein and the guide RNA do not naturally occur together.

Another representative, not limited, system is described by Frendewey, et al., U.S. Pat. No. 9,288,208 and comprises an in vitro method for modifying a genome at a genomic locus of interest in a mouse ES cell, comprising: contacting the mouse ES cell with a Cas9 protein, a CRISPR RNA that hybridizes to a CRISPR target sequence at the genomic locus of interest, a tracrRNA, and a large targeting vector (LTVEC) that is at least 10 kb in size and comprises an insert nucleic acid flanked by: (i) a 5′ homology arm that is homologous to a 5′ target sequence at the genomic locus of interest; and (ii) a 3′ homology arm that is homologous to a 3′ target sequence at the genomic locus of interest, wherein following contacting the mouse ES cell with the Cas9 protein, the CRISPR RNA, and the tracrRNA in the presence of the LTVEC, the genome of the mouse ES cell is modified to comprise a targeted genetic modification comprising deletion of a region of the genomic locus of interest wherein the deletion is at least 30 kb and/or insertion of the insert nucleic acid at the genomic locus of interest wherein the insertion is at least 30 kb. Other representative, but not limited, systems are described by WO 2014/089541 which is incorporated by reference and comprises methods for treating or repairing genes associated with hemophilia A. The methods of the present invention, which identify or quantify, corrections or repairs to genes are particular useful when used in conjunction with the genome or gene editing procedures described below because molecular combing easily detects genetic corrections and repaired genes provided made by these methods.

The F8 gene, located on the X chromosome, encodes a coagulation factor (Factor VIII) involved in the coagulation cascade that leads to clotting. Factor VIII is chiefly made by cells in the liver, and circulates in the bloodstream in an inactive form, bound to von Willebrand factor. Upon injury, FVIII is activated. The activated protein (FVIIIa) interacts with coagulation factor IX, leading to clotting. Mutations in the F8 gene cause hemophilia A (HA). Over 2,100 mutations in this gene have been identified, including point mutations, deletions, and insertion. One of the most common mutations includes inversion of intron 22, which leads to a severe type of HA. Mutations in F8 can lead to the production of an abnormally functioning FVIII protein or a reduced or absent amount of circulating FVIII protein, leading to the reduction of or absence of the ability to clot in response to injury. In one aspect, the present invention is directed to the targeting and repair of F8 gene mutations in a subject suffering from hemophilia A using the methods described herein. Approximately 98% of patients with a diagnosis of hemophilia A are found to have a mutation in the F8 gene (i.e., intron 1 and 22 inversions, point mutations, insertions, and deletions).

Such a method may comprise introducing into a cell of the subject one or more isolated nucleic acids encoding a nuclease that targets a portion of an F8 gene containing a mutation that causes hemophilia A, wherein the nuclease creates a double stranded break in the F8 gene; and an isolated nucleic acid comprising a donor sequence comprising (i) a nucleic acid encoding a truncated FVIII polypeptide or (ii) a native F8 3′ splice acceptor site operably linked to a nucleic acid encoding a truncated FVIII polypeptide, wherein the nucleic acid comprising the (i) nucleic acid encoding a truncated FVIII polypeptide or (ii) native F8 3′ splice acceptor site operably linked to a nucleic acid encoding a truncated FVIII polypeptide is flanked by nucleic acid sequences homologous to the nucleic acid sequences upstream and downstream of the double stranded break in the DNA, and wherein the resultant repaired gene, upon expression, confers improved coagulation functionality to the encoded FVIII protein of the subject compared to the non-repaired F8 gene. Such a method may also involve inducing immune tolerance to a FVIII replacement product ((r)FVIII) in a subject having a FVIII deficiency and who will be administered, is being administered, or has been administered a (r)FVIII product comprising introducing into a cell of the subject one or more nucleic acids encoding a nuclease that targets a portion of the F8 gene containing a mutation that causes hemophilia A, wherein the nuclease creates a double stranded break in the F8 gene; and an isolated nucleic acid comprising a donor sequence comprising (i) a nucleic acid encoding a truncated FVIII polypeptide or (ii) a native F8 3′ splice acceptor site operably linked to a nucleic acid encoding a truncated FVIII polypeptide, wherein the nucleic acid comprising the (i) nucleic acid encoding a truncated FVIII polypeptide or (ii) native F8 3′ splice acceptor site operably linked to a nucleic acid encoding a truncated FVIII polypeptide is flanked by nucleic acid sequences homologous to the nucleic acid sequences upstream and downstream of the double stranded break in the DNA, and wherein the repaired gene, upon expression, provides for the induction of immune tolerance to an administered replacement FVIII protein product. Either of these methods may employ a nuclease that is a zinc finger nuclease (ZFN), Transcription Activator-Like Effector Nuclease (TALEN), or a CRISPR (Clustered Regularly Interspaced Short Palindromic Repeats)-associated (Cas) nuclease. Both of these methods may use a nuclease that intron 22 of the F8 gene, that targets intron 1 of the F8 gene, that targets the exon 22/intron 22 junction, or that targets the exon 1/intron 1 junction. Either of these methods may target an F8 mutation that comprises a mutation that is an intron 22 inversion.

Another representative method that is advantageously practiced with the molecular combing steps of the invention is a method described by an incorporated by reference to WO2015089465 which involves genome or gene editing of polynucleotides comprising the genes of persistent viruses such as hepatitis B virus. Such viruses persist due to integration of a virus into a host's genome and/or by maintenance of an episomal form (e.g. hepatitis B virus, HBV, which maintains extraordinary persistence in the nucleus of human hepatocytes by means of a long-lived episomal double-stranded DNA form called covalent closed circular DNA, or cccDNA). It has been shown that it is possible to directly cleave and reduce the abundance of this episomal form of the virus (cccDNA: a dsDNA structure that arises during the propagation of HBV in the cell nucleus and can remain permanently present in infected subjects).

The method involves modifying an organism or a non-human organism by manipulation of a target hepatitis B virus (HBV) sequence in a genomic locus of interest comprising delivering a non-naturally occurring or engineered composition comprising: A)—I. a CRISPR-Cas system RNA polynucleotide sequence, wherein the polynucleotide sequence comprises: (a) a guide sequence capable of hybridizing to a target HBV sequence in a eukaryotic cell, (b) a tracr mate sequence, and (c) a tracr sequence, and II. a polynucleotide sequence encoding a CRISPR enzyme, optionally comprising at least one or more nuclear localization sequences, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target HBV sequence, and wherein the CRISPR complex comprises the CRISPR enzyme complexed with (1) the guide sequence that is hybridized or hybridizable to the target HBV sequence, and (2) the tracr mate sequence that is hybridized or hybridizable to the tracr sequence and the polynucleotide sequence encoding a CRISPR enzyme is DNA or RNA, or (B) I. polynucleotides comprising: (a) a guide sequence capable of hybridizing to a target HBV sequence in a eukaryotic cell, and (b) at least one or more tracr mate sequences, II. a polynucleotide sequence encoding a CRISPR enzyme, and III. a polynucleotide sequence comprising a tracr sequence, wherein when transcribed, the tracr mate sequence hybridizes to the tracr sequence and the guide sequence directs sequence-specific binding of a CRISPR complex to the target HBV sequence, and wherein the CRISPR complex comprises the CRISPR enzyme complexed with (1) the guide sequence that is hybridized or hybridizable to the target HBV sequence, and (2) the tracr mate sequence that is hybridized or hybridizable to the tracr sequence, and the polynucleotide sequence encoding a CRISPR enzyme is DNA or RNA.

The molecular combing steps of the invention may be used in conjunction with therapeutic genome or gene editing techniques described by WO 2014/165825 which are incorporated by reference. These techniques comprise a method for altering a target polynucleotide sequence in a cell comprising contacting the polynucleotide sequence with a clustered regularly interspaced short palindromic repeats-associated (Cas) protein and from one to two ribonucleic acids, wherein the ribonucleic acids direct Cas protein to and hybridize to a target motif of the target polynucleotide sequence, wherein the target polynucleotide sequence is cleaved, and wherein the efficiency of alteration of cells that express Cas protein is from about 0, 10, 20, 30, 40, 50, 60, 79, 80, 90 to about 100%. This method may be used for treating or preventing a disorder associated with expression of one or more polynucleotide sequence(s) in a subject and may involve (a) altering a target polynucleotide sequence in a cell ex vivo by contacting the polynucleotide sequence with a clustered regularly interspaced short palindromic repeats-associated (Cas) protein and from one to two ribonucleic acids, wherein the ribonucleic acids direct Cas protein to and hybridize to a target motif of the target polynucleotide sequence, wherein the target polynucleotide sequence is cleaved, and wherein the efficiency of alteration of cells that express Cas protein is from about 0, 10, 20, 30, 40, 50, 60, 79, 80, 90 to about 100%, and (b) introducing the cell into the subject, thereby treating or preventing a disorder associated with expression of the polynucleotide sequence. Such methods may be practiced using a human pluripotent cell, a primary human cell, or a non-transformed human cell.

The invention may also be practiced in combination with the genome or gene editing techniques described by US 20150056705 A1. These may include a method of modifying the expression of an endogenous gene in a cell, the method comprising the steps of: administering to the cell a first nucleic add molecule comprising a single guide RNA that recognizes a target site in the endogenous gene and a second nucleic acid molecule that encodes a functional domain, wherein the functional domain associates with the single guide RNA on the target site, thereby modifying the expression of the endogenous gene; optionally where the functional domain is selected from the group consisting of a transcriptional activation domain, a transcriptional repression domain and a nuclease domain or where the functional domain is a TypeIIS restriction enzyme nuclease domain or a Cas protein.

None of these patents or patent applications contemplated applying CRISPR-Cas9 like, ZNF, or TALEN mediated genomic or gene editing in combination with molecular combing, nor did they recognize the advantages attained by this combination, such as the avoidance of bias and the improved efficiency provided by a single assay as disclosed herein.

Nuclease Induced-Gene Editing Events

Based on the ability of modified nuclease to create site-specific DSB, it is possible to harness the cell's endogenous machinery in order to engineer a wide variety of genomic alterations in a site specific manner. These genomic alterations include Gene knockout/mutation, Gene correction, Gene deletion and Gene insertion. These procedures are effectively used in combination with molecular combing.

Gene Knockout/Mutation

This simplest form of gene editing utilizes the error-prone nature of NHEJ at the target site. This process is active during all stages of the cell cycle and repair DNA with a high frequency of mutagenesis resulting in the formation of indels at the site of the break (Chapman, Taylor et al. 2012).

When the nuclease target site is placed in the coding region of a gene, the resulting indels will often cause frameshifts and, in most of the case, to subsequent gene knockout. However, in diseases such as Duchenne muscular dystrophy (DMD), where gene deletions result in frameshifts and subsequent loss of protein function, targeted NHEJ-induced indels can be used to restore the correct reading frame of the gene (Ousterout, Perez-Pinera et al. 2013). Moreover, gene disruption may be used to correct dominant gain-of-function mutations and thus used therapeutic treatment as it has been shown in Huntington's disease (Aronin and DiFiglia 2014) or dominant dystrophic epidermolysis bullosa (Shinkuma, Guo et al. 2016). In contrast, therapeutic effect can be also achieved to remove the normal function. This approach is typically used to target the host viral receptors to prevent viral infection as it the case for the treatment of HIV, in which knockout of CCR5, the major HIV co-receptor, prohibits viral infection of modified T cells (Gu 2015). Finally, rather than directly targeting the human genome, knockout of critical genes in invading bacteria or DNA-based viruses could serve as effective anti-microbial treatments (Beisel, Gomaa et al. 2014; White, Hu et al. 2015)

Gene Correction

As targeted DSBs can induce precise gene editing by stimulating HR with an exogenously supplied donor template, any sequence differences present in the donor template can thus be incorporated into the endogenous locus to correct disease-causing mutations, as has been demonstrated in numerous studies, especially in the treatment of primary immunodeficiency disorders (Cicalese and Aiuti 2015).

Gene Deletion

It is also possible to delete large segments of DNA by flanking the targeted sequence with two DSBs by simultaneously introducing of two targeted modified nucleases. The size of the resulting genomic deletions can reach several megabases (Sollu, Pars et al. 2010; Canver, Bauer et al. 2014). This approach could be useful for therapeutic strategies that may require the removal of an entire genomic element, such as the intronic sequence in the CEP290 gene containing a frequent mutation that creates an aberrant spice site disrupting the coding sequence in Leber Congenital Amaurosis (Maeder and Gersbach 2016).

Gene Insertion

The use of a DNA donor template, in which the desired genetic insert is flanked by homology sequences identical to the nuclease cut site, enables site-specific DNA insertion through DSB-induced HR (Moehle, Rock et al. 2007). An alternative mechanism for targeted transgene insertion is to use nuclease-induced DSBs to create compatible overhangs on the donor DNA and the endogenous site, leading to NHEJ-mediated ligation of the insert DNA sequence directly into the target locus (Maresca, Lin et al. 2013). In the case where a wild type copy of a gene is inserted into the endogenous mutated locus, the main advantage is that the expression is controlled by the natural regulatory elements and will reduce the risk associated with random transgene insertion as it was observed in the early clinical trials with retroviral vector (For review (Baum, Modlich et al. 2011).

Assessment of the Efficiency of Modified Nucleases (On-Target)

In order detect and quantify the efficiency of gene editing mediated by modified nucleases, both immediately after treatment and as follow-up on gene-edited cells in vivo (for example, using blood samples from patients in clinical studies), numerous technologies have been developed: phenotype selection, restriction site selection, PAGE-based genotyping method, enzymatic mismatch cleavage-based assays, subcloning of affected genomic locus, high-resolution melting curve (HRM) analysis, Next gene sequencing (NGS) and droplet digital PCR (ddPCR), see (Shendure and Ji 2008) (Hindson, Chevillet et al. 2013) which are incorporated by reference.

Phenotype Selection

Phenotype selection is based on the fact that substances (molecules, peptides . . . ) or a treatment (RNAi, gene editing . . . ) alter the phenotype of a cell or an organism in a desired manner. This approach has been successfully used to characterize the effect of ZFN on zebrafish (Doyon, McCammon et al. 2008). The major limitation of phenotype selection relies on the fact that many gene do not show an apparent phenotype after treatment.

Restriction Site Selection

Restriction site selection requires a specific restriction site within the region of detection. Upon nuclease-mediated modification, a gene or its fragment may lose or acquire the recognition site for the restriction enzyme, leading to a change in the restriction pattern as it has been shown in TALENs-targeted zebrafish (Huang, Xiao et al. 2011). The use of this method is restricted to known mutation that can be targeted by site restriction enzyme.

PAGE-Based Genotyping Method

In this approach, the PCR-amplified genomic regions spanning the mutagenesis site undergo a brief denaturation and annealing cycle. Then, PCR fragments from genetically modified individuals, which contain a mixture of Indel mutations and wild type alleles, will form heteroduplex and homoduplex DNAs. Due to the existence of an open angle between matched and mismatched DNA strands caused by Indel mutations, heteroduplex DNA generally migrate at a significantly slower rate than homoduplex DNA in a native Polyacrylamide Gel Electrophoresis (PAGE), thus making it a useful tool to screen founders harboring mutations (Zhu, Xu et al. 2014). However, this is not a high-throughput approach, it is time-consuming and it does not provide any exact information about the mutations, although it is affordable in terms of feasibility and costs.

Enzymatic Mismatch Cleavage-Based Assays

To identify unknown mutations, the identification of heteroduplex DNA formed after melting and hybridizing mutant and wild type alleles is widely used. The identification of heteroduplex DNA can be done with chemicals (Bhattacharyya and Lilley 1989), enzymes (Mashal, Koontz et al. 1995; Taylor and Deeble 1999), or proteins that bind mismatches (Wagner, Debbie et al. 1995). The enzyme mismatch cleavage (EMC) method takes advantages of enzymes able to cleave heteroduplex DNA at mismatches formed by single or multiple nucleotides. The first enzymes used for EMC were bacteriophage resolvases such as T4E7 and T7E1 (Mashal, Koontz et al. 1995). However, this method work with moderate success because deletions are cleaved more efficiently than single base mutations (Mashal, Koontz et al. 1995).

A second generation of single-strand specific endonucleases of the S1 nuclease family such as CEL (CELII nuclease is commercialized under the brand Surveyor®) (Qiu, Shandilya et al. 2004) and ENDO (Triques, Piednoir et al. 2008) has been used more recently for mutation detection. The Surveyor-based EMC assay is used commonly to scan mutations induced by engineered nucleases (Qiu, Shandilya et al. 2004; Guschin, Waite et al. 2010).

EMC assays are cost-effective methods that can be performed with the use of simple laboratory setups but its sensitivity is limited (>1%) and quantification is comparatively imprecise (Vouillot, Thelie et al. 2015).

Subcloning of the Targeted Region

This strategy consists of subcloning of the affected genomic locus by PCR followed by Sanger sequencing and subsequent counting of modified alleles (Perez, Wang et al. 2008). This method can be performed without special equipment but is quite laborious, time-consuming and expensive. Moreover, sensitivity and accuracy directly depend on the number of cloned sequenced (around sequencing of 300 clones have to be analyzed to reach a sensitivity of 1%) and can be biased by the use of the amplification step.

High-Resolution Melting Curve (HRM) Analysis

High Resolution Melting Analysis (HRM) is a post-PCR method. The region of interest within the DNA sequence is first amplified using PCR in presence of saturation intercalating dyes that fluoresce only in the presence of double stranded DNA. As the amplicon concentration in the reaction tube increases during the PCR cycles, the fluorescence exhibited by the double stranded amplified product also increases. After the PCR, the amplicon DNA is heated gradually from around 50° C. up to around 95° C. When the melting temperature of the amplicon is reached, the double stranded DNA melts apart and the fluorescence fades away. This observation is plotted showing the level of fluorescence vs the temperature, generating a Melting Curve. Even a single base change in the sample DNA sequence causes differences in the HRM curve. Since different genetic sequences melt at slightly different rates, they can be viewed, compared, and detected using these curves. This approach has been used for evaluation of gene editing efficiency (Thomas, Percival et al. 2014; D'Agostino, Locascio et al. 2016). However, as NHEJ repair mechanism may result in a diverse pattern of Indels, multiple PCR products will be generated, which precludes the demarcation of a defined second melting curve and thus prevents exact quantification.

Next Gene Sequencing

There are a number of different NGS platforms using different sequencing technologies that allow massively sequencing of millions of small fragments of DNA in parallel. This technology is the most widely used approach to evaluate the efficiency of gene editing, for example, Bell, Magor et al. 2014; Guell, Yang et al. 2014; Hendel, Kildebeck et al. 2014; Schmid-Burgk, Schmidt et al. 2014. The major advantage of this method is the possibility to simultaneously analyze the on-target and the potential off-target sites. However, NGS sensitivity depends on four variables (depending on the sequencing technologies). First, it depends on the amount of genomic DNA (gDNA) used for amplification of the target locus (100 ng of gDNA would confer a sensitivity of 0.02%). Second, NGS sensitivity is contingent of the library size and the number of read counts (15 000 reads are theoretically required for a sensitivity of 0.02%). Third, it also depends on the intrinsic rate of NGS errors that can interfere with the analysis. Fourth, the read-length limitations of some platforms do not allow analysis of long arms of homology that drive more efficient HR, especially in the case of gene insertion.

Droplet Digital PCR

Droplet digital PCR (ddPCR) is a sensitive method enabling the accurate quantification of a target nucleic acid sequence (Vogelstein and Kinzler 1999; Pinheiro, Coleman et al. 2012). In this method, individual DNA molecules from a sample are captured within water-in-oil droplet partitions (Pinheiro, Coleman et al. 2012). Droplets containing mutant or wild-type allele are discriminated using two color-fluorescent TaqMan probes and the numbers of target DNA copies are counted at the end point of PCR (Vogelstein and Kinzler 1999). Some specific modification of ddPCR have been done to assess gene-editing frequencies that combines high sensitivity (<0.2%) with excellent accuracy (Mock, Hauber et al. 2016). The limitations of the ddPCR are identical to the classical PCR: dependent on the sequence information, limited amplification size, error rated during the amplification, sensitivity to inhibitors, limits on exponential amplification and artefacts, and sensible to contamination.

Detection and Quantification of Off-Target Events

One potential complication of the gene editing tools is that the modified nuclease will create other, unwanted genomic changes. This “off-target” activity of the modified nucleases occurs fundamentally because they are able to bind to sequences other than the intended DNA target. The most common manifestation of the off-target activity is small indels du to NHEJ. However, gross chromosomal rearrangements are the most concerning type of off-activity effects since they are most clearly associated with malignant transformation. Genomic alterations reported in the literature include incorporation into the genome of exogenously supplied DNA such as a donor DNA template or contaminant bacterial DNA remaining after plasmid production (Hendel, Kildebeck et al. 2014), deletion of large region of chromosomal sequences (Cradick, Fine et al. 2013; Mussolino, Alzubi et al. 2014), duplications and inversions (Lee, Kweon et al. 2012), chromosomal translocations (Torres, Martin et al. 2014) and sequence insertion from alternate locations in the genome (Hendel, Kildebeck et al. 2014).

Functional Assays

There are several assays that can measure the functional toxicity of modified nuclease expression without having to predict potential off-target sites. These assays include induction of cellular apoptosis (Mussolino, Alzubi et al. 2014), modification of replicative parameters compared to cells not expressing the modified nuclease (Pruett-Miller, Connelly et al. 2008; Maeder, Linder et al. 2013), soft agar transformation and clonal expansion assays (Porter, Baker et al. 2014).

Detection of Off-Target Sites

There are several in vitro and cellular assays to detect the most probable off-target sites. For example, in vitro binding of modified nucleases to oligonucleotides can be used identify sequences that are to be cleaved in vitro and then these sequences can be searched in the genome for exact matches to those sequences (Pattanayak, Ramirez et al. 2011; Pattanayak, Lin et al. 2013). Another approach consists of chromatin immunoprecipitation to pull down the modified nucleases activity, followed by sequencing the DNA fragments to which the nuclease is bound and mapping those fragments to the genome (Kuscu, Arslan et al. 2014; Wu, Scott et al. 2014).

Unbiased assays have been developed. They rely on trapping integrative-deficient lentivirus or adenovirus (IDLV capture method) (Gabriel, Lombardo et al. 2011; Wang, Wang et al. 2015; Osborn, Webber et al. 2016) or small-modified double strand oligonucleotides (dsODN; GUIDE-Seq method) (Tsai, Zheng et al. 2015) at the site of DSB and genomic locations are identified by LAM-PCR (IDLV-Capture) or tag-specific amplification (GUIDE-Seq) and high-throughput sequencing.

Nevertheless, all these methods are technically challenging. For example, GUIDE-Seq technology requires high level of transfection efficiency on the target cells, which limit the use of this method in some cell types. Moreover, some of these technologies such as immunoprecipitation may lead with very high false-positive detection rates (Kuscu, Arslan et al. 2014; Wu, Scott et al. 2014). The sensitivity of these methods to detect low level of off-target events might also be low (Gabriel, Lombardo et al. 2011).

An alternative method consists of sequencing the whole genome before and after gene editing. In that way, off-target sites can be determined by a simple analysis of the new mutations that have been generated outside the intended locus, as compared with the original population (Smith, Gore et al. 2014; Iyer, Shen et al. 2015). However, whole genome sequencing, which only detects high frequency of off-target sites, lacks sensitivity required to detect off-target sites in bulk population (Veres, Gosis et al. 2014).

Prediction of Off-Target Site Locations

Theoretically the entire genome could be considered as potential off-target sites. However, modified nuclease-induced off-target events are presumed to be a direct result of the nuclease binding to a DNA sequence with some level of homology with the intended targeted site. Therefore, modified nuclease tend to induce off-target event at certain hot-spot locations that are consistent in frequency and location for a given modified in a given cell type or in different cell type of the same species (Fu, Foden et al. 2013).

Algorithms have been generated using the data generated by different research groups on the off-target cleavage of CRISPR-Cas9 in order to predict the most probable off-target sites. These algorithms include the Cas-OFFinder (Bae, Park et al. 2014), the CasFinder (Aach, Mali et al. 2014), the CRISPR Design tool (Hsu, Scott et al. 2013), the E-CRISPR (Heigwer, Kerr et al. 2014) and the Breaking-cas (Oliveros, Franch et al. 2016) and many others. However, different factors (position of the mismatch in the gRNA, genomic or epigenomic context, . . . ) might affect the cleavage frequency making difficult the development of an algorithm capable of identifying all potential off-target sites.

There is a need for more efficient and accurate methods for identifying, screening and selecting polynucleotides containing genome modifications or edits and also for selecting the most appropriate genome editing system that induces the expected genome modification(s) or gene editing events. The methods described above each have one or more limitations such as those described above. Significant limitations to present methods include that existing methods are indirect. They do need pre-analytical steps such as gene amplification, library preparation, and/or subcloning. Due to the need for these pre-analytical steps, prior methods are often subject to significant bias making the precise quantification of genome modifications or gene editing events difficult. Most of the prior art methods are inefficient and incapable of detecting on-target and off-target methods in a single assay. Some prior methods are limited to detection of known mutations or variations in a polynucleotide and fail to detect off-target events. Many of the prior methods have limited sensitivity and do not detect or quantify rare genomic modification or gene editing events.

The present invention involves genetic modifications of the targeted cellular genomic DNA. The modifications include deletions, duplications, amplifications, translocations, insertions or inversions of part or all of the gene sequence including but not limited to the coding region and to the regulatory elements sequences, etc.

The standard reference acid nucleic sequences correspond to the wild type nucleic acid sequences or to selected mutated sequences of interest such as a predetermined nucleic acid sequence.

### BRIEF DESCRIPTION OF THE INVENTION

In view of the limitations and drawbacks for existing methods described above, the inventors diligently sought ways to improve the efficiency and accuracy of detecting genome modifications and gene editing events. The molecular combing (“MC”) based methods disclosed herein overcome limitations with prior methods of accurately detecting genome editing events such as those performed with CRISPR-Cas9 techniques or with other genome editing procedures. The molecular combing-based methods according to the invention can detect and quantify rare events that occur during genome or gene editing procedures.

These methods do not require pre-analytical steps and thus avoid the introduction of bias attributable to these pre-analytical steps. The method of the invention by counting large numbers of individual genome or gene editing events makes possible very precise quantification of such events including rare events not detectable using current methodologies. The use of GMC (“Genomic Morse Code”) permits the detection of both expected gene editing events as well as rare or unexpected editing events in the region covered by the GMC as shown below in the Examples and in FIGS. 2D-2G. The addition of GMC covering potential off-target events, molecular combing allows one to detect On- and Off-target events in a single assay. This assay directly inspects and counts each molecule without the bias introduced by the pre-analytical steps required by existing detection methods, thus providing a more efficient and accurate method for detection and quantification of genome and gene editing events.

### BRIEF DESCRIPTION OF THE DRAWINGS

FIG. 1A. Schematic representation of the genomic structure of recombinant HSV-1 (rHSV-1) and of the different hybridization patterns that might be observed in control and I-SceI-treated rHSV-1 samples (biotin labelled-rHSV-1 probes are represented in white boxes; Alexa Fluor® 488-labelled LacZ probes are depicted in grey boxes). The overall structure of the rHSV-1 genome is shown with unique long (U_{L}) and short (U_{S}) regions and the TR_{L}/TR_{S} and IR_{L}/IRₛ repeats. An expression cassette containing the cytomegalovirus (CMV) promoter and the LacZ coding sequence was inserted in the major latency-associated (LAT) genes. The I-SceI target site was cloned between the CMV promoter and the LacZ gene. The minimal requirement hybridization patterns as defined in the “Analysis of HSV-1 detected signals” section are also indicated just above the complete signal.

FIG. 1B. Several representative linear hybridization chains showing example of intact or I-SceI-digested/broken rHSV-1 DNA molecules (White: Alexa Fluor® 594-fluorescence: rHSV-1 probes; grey: Alexa Fluor® 488-fluorescence: LacZ probe).

FIG. 1C. Histogram showing the frequency of intact (white bars) and I-SceI-digested/broken (grey bars) rHSV-1 DNA molecules in both control and I-SceI-treated rHSV-1 samples.

FIG. 1D. Genomic structure of rHSV-1 (see FIG. 1A) and primer pairs used for detection of different regions of the rHSV-1 genome as precised in Table A.

FIG. 1E. Example of semi-quantitative PCR results on in vitro I-SceI-treated and control rHSV-1 DNA. The I-SceI-untreated rHSV-1 used as control (−) and the I-SceI-treated rHSV-1 samples (+) are amplified by PCR using target-specific primers as described in Table A. H₂O and pCLS0126 (a viral vector with the pCMV-LacZ gene in the LAT gene) are used as negative and positive PCR control, respectively. In this example, no PCR product is observed in the negative control and a specific amplification product is detected with the positive control and with I-SceI-untreated rHSV1 whatever the primer pairs used and the dilution (except for 1:1000 which is below to the detectability limit). In contrast, for the I-SceI-treated, no amplification product was observed with both Sce1a and Sce1b primer pairs that overlap the 1-SceI target site.

FIG. 2A. Schematic representation of the BRCA1 GMC v5.2 used to evaluate the efficiency of CRISPR-Cas9 RNA-guided 6.5 kb-deletion. The complete BRCA1 GMC v5.2 covers a region of approximatively 200 kb and is composed of 16 fluorescent probes (B, a, b, c, d, e, f, g, h, I, j, k, l, m, n and R) that are labelled with different haptens as described in “Synthesis and labelling of BRCA Probes” (aminoDIG9-labelled probes are represented by black boxes, Fluo- and Biot-labelled probes are depicted by grey and white boxes, respectively). The region encoding BRCA1 (81.2 kb) is composed of 8 probes (a-h) and its 5′-upstream region is composed of 6 probes (i-n) including the BRCA1 pseudogene, ΨBRCA1 (j-k). The probes B and R located at each extremity of the BRCA1 GMC v5.2 are used as anchoring probes to demarcate the region of interest. The relative positions of the BRCA1 exons are shown above the schematic representation of the BRCA1 GMC v5.2.

FIG. 2B. CRISPR-Cas9 targeting of the BRCA1 gene. gRNA sequences were designed to bind sequences flanking the BRCA1 genomic region covered by the apparent blue b probe of the BRCA1 GMC v5.2. Grey arrows indicate the relative position of gRNA (as specified in Table B) that were designed to bind sequences flanking the BRCA1 genomic region covered by the 6.5 kb-apparent blue b probe (GRCh37/hg19 sequence: chr17: 41,205,246-41,211,745). Black arrows shows relative position of PCR primers used for the detection of the 6.5-kb deletion as indicated in Table C. Plain lines represent the region deleted region for each gRNA combination as specified in Table D and the size of the expected PCR products obtained after gene editing is indicated.

FIG. 2C. Agarose gel electrophoresis (2%) of amplification products of the CRISPR-Cas9-targeted BRCA1 region (GRCh37/hg19 sequence: chr17: 41,205,246-41,211,745) in transfected HEK293 cells (line 1-9 as specified in Table D) and in isogenic control (line 10) using the BRCA-Left-PCR-F and BRCA-Right-PCR-R (upper panel) and BRCA-Left-PCR-F and BRCA-Left-PCR-R (lower panel) primers pairs.

FIG. 2D. Examples of normal and edited BRCA1 fluorescent arrays on combed DNA extracted from HEK293 cells transfected with the Left-gRNA7+BRCA-Right-gRNA4 (upper panel), Left-gRNA7+BRCA-Right-gRNA9 (middle panel) and Left-gRNA7+BRCA-Right-gRNA12 (lower panel) gRNA pairs. Schematic representation of the normal BRCA1 fluorescent array is indicated (aminoDIG9-labelled probes are represented by black boxes, Fluo- and Biot-labelled probes are depicted by grey and white boxes, respectively).

FIG. 2E. Histogram of the distribution normal and edited BRCA1 fluorescent arrays in isogenic HEK293 cells (control) and in HEK293 cells transfected with the Left-gRNA7+BRCA-Right-gRNA4, Left-gRNA7+BRCA-Right-gRNA9 and Left-gRNA7+BRCA-Right-gRNA12 gRNA pairs. Hybridization signals were selected and analyzed as described in the “Example 2” section. In this example, a total of hybridization signals comprising between 238 and 740 fluorescent signals per condition were identified and classified. No edited BRCA1 gene was detected in the isogenic HEK293 control cells whereas 10.5%, 11.1% and 6.5% of edited BRCA1 gene (where sequence b has been deleted) have been quantified in transfected HEK293 cells with the Left-gRNA7+BRCA-Right-gRNA4, Left-gRNA7+BRCA-Right-gRNA9 and Left-gRNA7+BRCA-Right-gRNA12 gRNA pairs, respectively. Error bars represent 95% confidence intervals. Proportions with stars are significantly different at adjusted level alpha=0.05 (*) 0.01 (**) 0.001 (***).

FIG. 2F. Detection of other large rearrangements in the BRCA1 gene induced by the designed CRISPR-Cas9 system. Examples of a duplication/inversion in the BRCA1 gene detected in HEK293 cells transfected with the Left-gRNA7+BRCA-Right-gRNA4 gRNA pair. Schematic representation of the hybridization patterns corresponding of the potential duplication/inversion of the BRCA1 gene is indicated (aminoDIG9-labelled probes are represented by black boxes, Fluo- and Biot-labelled probes are depicted by grey and white boxes, respectively). The hatched boxes represents the region of BRCA1 GMC v5.2 that has been deleted (blue B and green a probes) in these examples. The regions of the BRCA1 GMC v5.2 that are indicated between brackets correspond to regions that have not been observed in the fluorescent arrays probably due to random breakage of DNA molecules during the Molecular Combing process. The breakpoint of the duplication/inversion is located within the sequence of the apparent blue b probe (indicated by the cross).

FIG. 2G. Histogram of the distribution rearranged BRCA1 fluorescent arrays in isogenic HEK293 cells (control) and in HEK293 cells transfected with the Left-gRNA7+BRCA-Right-gRNA4, Left-gRNA7+BRCA-Right-gRNA9 and Left-gRNA7+BRCA-Right-gRNA12 gRNA pairs. Hybridization signals were selected and analyzed as described in the “Example 2” section. In this example, a total of hybridization signals comprising between 238 and 740 fluorescent signals per condition were identified and classified. 0.9%, 3.8%, 2.5% and 1.6% of rearranged BRCA1 gene have been quantified in isogenic HEK293 control cells and in transfected HEK293 cells with the Left-gRNA7+BRCA-Right-gRNA4, Left-gRNA7+BRCA-Right-gRNA9 and Left-gRNA7+BRCA-Right-gRNA12 gRNA pairs, respectively. Error bars represent 95% confidence intervals. Proportions with stars are significantly different at adjusted level alpha=0.05 (*) 0.01 (**) 0.001 (***).

FIG. 3A. Histogram of the distribution of deletion events in the BRCA1 gene measured by ddPCR in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs. The genomic DNAs extracted from isogenic (control) or transfected HEK293 cells were analyzed in triplicates or quadruplicates as described in the “Example 2” section. Because of threshold choice during ddPCR analysis, few deletion events were artefactual detected in isogenic HEK293 cells (control). The mean value of these events was subtracted from the count of deletions observed in transfected cells. A total number of events (normal alleles plus deletions) between 1592 and 2656 were measured for each sample. 14.3%, 12.0% and 7.9% of edited BRCA1 gene (6.5 kb deletion) have been quantified in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs, respectively. Error bars represent standard deviations.

FIG. 3B. Histogram of the distribution of deletion events in the BRCA1 gene measured by targeted-NGS in isogenic HEK293 cells (control) and in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs. The genomic DNAs extracted from isogenic (control) or transfected HEK293 cells were analyzed in duplicates as described in the “Example 2” section. A total number of events (normal alleles, deletions and rearrangements) between 1394 and 2086 were measured for each sample. One deletion event was detected in the isogenic HEK293 control cells whereas 1.3%, 1.3% and 1.0% of edited BRCA1 gene have been quantified in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs, respectively. Results are presented as the mean of duplicated experiments.

FIG. 3C. Histogram of the distribution of rearranged BRCA1 gene measured by targeted-NGS in isogenic HEK293 cells (control) and in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs. The genomic DNAs extracted from isogenic (control) or transfected HEK293 cells were analyzed in duplicates as described in the “Example 2” section. A total number of events (normal alleles, deletions and rearrangements) between 1394 and 2086 were measured for each sample. No rearranged BRCA1 gene was detected in the isogenic HEK293 control cells whereas 2.6%, 2% and 1.1% of rearranged BRCA1 gene have been quantified in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs, respectively. Results are presented as the mean of duplicated experiments.

### DETAILED DESCRIPTION OF THE INVENTION

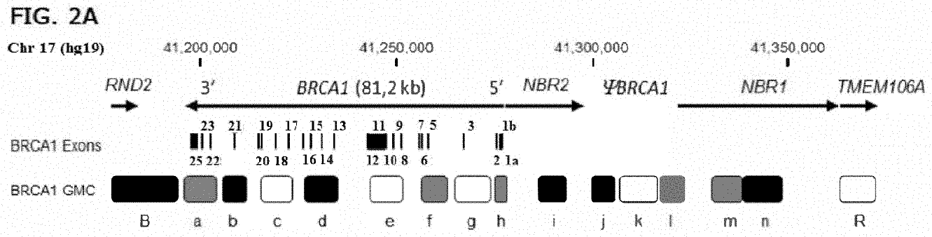

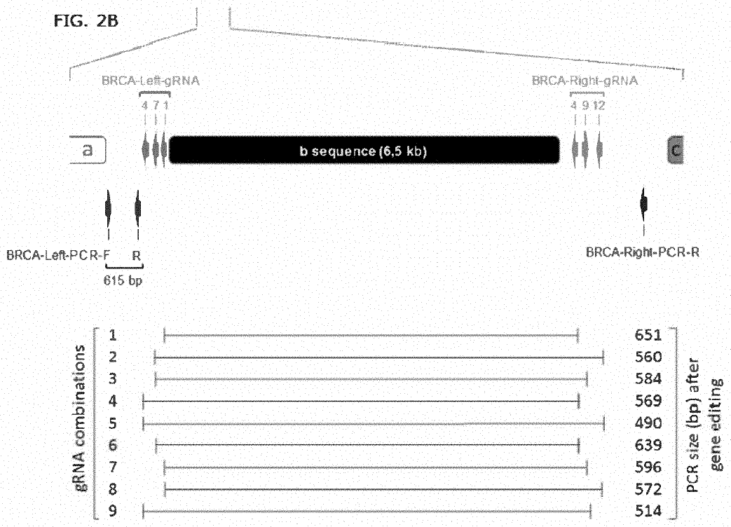

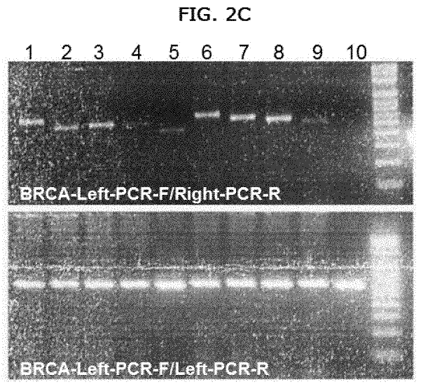

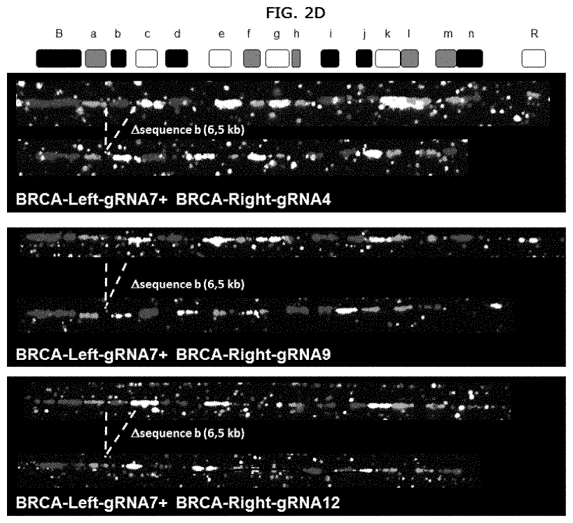

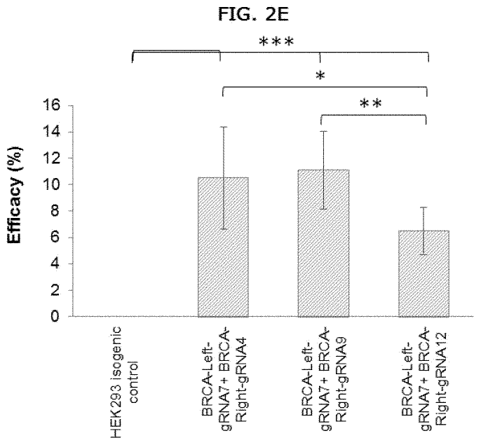

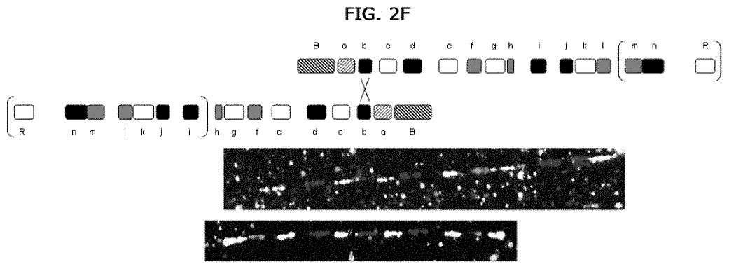

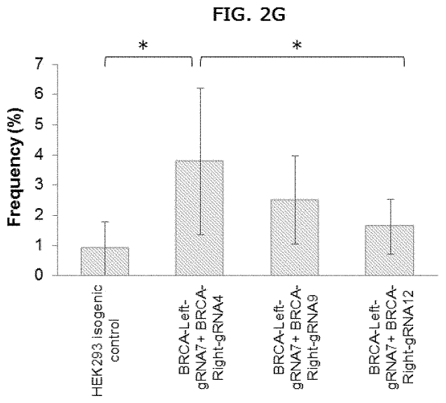

As explained above, the Molecular Combing based methods of the invention do not require pre-analytical steps and thus avoid the introduction of bias attributable to these pre-analytical steps and permit the detection of both expected gene editing events as well as rare or unexpected gene editing events as shown below in the Examples and in FIGS. 2D-2G. The gene or genome editing genome may involve a complete gene or genome or a fragment of gene or genome. These events can be detected in a single assay that directly inspects and counts each molecule without the bias introduced by pre-analytical steps. The surprising advantages of a method that combines molecular combing with genome or gene editing using CRISPR have not been previously recognized.

The present invention provides a new method for quality control of editing procedures using modified nucleases using Molecular Combing. The method comprises at least two, preferably at least three steps characterized by, first, the modification of the polynucleotide(s) of interest by a modified nuclease, second the detection, the characterization and the quantification of the modified polynucleotide(s) by molecular combing comprising selected fluorescent polynucleotides and optionally, third, the comparison with one or more control samples, which have not been treated with the modified nuclease, to determine the efficacy and/or the specificity associated with the modified nuclease. Optionally, the modified polynucleotide(s) which have been detected during the molecular combing process allow selection of the most accurate and efficient modified nuclease for therapeutic applications, such as gene correction and gene modification. The method may also, optionally, comprise the use of at least one modified nuclease or multiple modified nucleases depending on the targeted region(s) in a polynucleotide of interest, such as a portion of the genome or a target gene.

The present invention is also directed to an alternative method that detects, in a biological sample of a patient treated with the selected modified nuclease, the genetic modifications induced by a selected modified nuclease in order to follow the treatment efficacy and safety. In this embodiment, the method comprises the following steps: first, the modification of the polynucleotide of interest by a modified nuclease and then by detecting, characterizing and quantifying the modified polynucleotide(s) by molecular combing, comprising selected fluorescent polynucleotides. In this embodiment, a comparison between the samples before and after the use of the selected modified nuclease may optionally be made, thus allowing a more accurate determination of the treatment efficacy and safety. Optionally, this method may comprise the use of multiple modified nucleases depending on the targeted genomic regions to be corrected or modified, such as target polynucleotide regions involved in polygenic diseases.

Genome or gene editing of particular genetic diseases or disorders that may be detected, characterized, or quantified according to the invention include, but are not limited to Achondroplasia, Alpha-1 Antitrypsin Deficiency, Antiphospholipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Facio-Scapulo-Humeral Dystrophy (FSHD), Familial Mediterranean Fever, Fragile X Syndrome, Gaucher Disease, Hemochromatosis, Hemophilia, Holoprosencephaly, Huntington's disease, Klinefelter syndrome, Leber Congenital Amaurosis, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Turner Syndrome, Velocardiofacial Syndrome, WAGR Syndrome, and Wilson Disease.

The method of the invention may be employed to detect, characterize, assess or quantify genome or gene editing events in a polynucleotide, genome, exon, intron, or gene of choice. Specific kinds of genes include, but are not limited to prokaryotic or eukaryotic genes or genomes, yeast or fungal genomes or genes, plant or algae genes, invertebrate or vertebrate genes, genes from fish, amphibians, reptiles, birds including chickens, turkeys and ducks, mammalian genes including those of domesticated animals, such as horses, cattle, cows, goats, sheep, llamas, camels, or pigs.

Such genes include any of the following a mammalian β globin gene (HBB), a gamma globin gene (HBG1), a B-cell lymphoma/leukemia 11A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (Htt) gene, a rhodopsin (RHO) gene, a Cystic Fibrosis Transmembrane Conductance Regulator (CFTR) gene, a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CIITA) gene, a dystrophin gene (DMD), a glucocorticoid receptor gene (GR), an IL2RG gene, a centrosomal protein of 290 kDa (CEP290), Double homeobox 4 (DUX4) and an RFX5 gene. Such genes also include a plant FAD2 gene, a plant FAD3 gene, a plant ZP15 gene, a plant KASII gene, a plant MDH gene, and a plant EPSPS gene.

Accordingly the invention is directed to a method for detecting, characterizing, quantifying or determining the efficiency of a gene or genome editing procedure or event comprising a step of Molecular Combing which is carried out as a step of stretching nucleic acid, extracted from any source to be assessed (from virus, bacteria to human through plants . . . ) to provide immobilized nucleic acids in linear and parallel strands (aligned nucleic acids). Molecular Combing is thus preferably performed with a controlled stretching factor (such as a meniscus as disclosed hereafter) formed on an appropriate surface (e.g., surface-treated glass slides). After stretching, it is possible to hybridize sequence-specific probes detectable for example by fluorescence microscopy (Lebofsky, Heilig et al. 2006). Thus, a particular nucleic acid sequence may be directly visualized on a single molecule level. The length of the fluorescent signals and/or their number, and/or their spacing on the slide provides a direct reading of the size and relative spacing of the probes.

Molecular combing is accordingly a technique enabling the direct visualization of individual nucleic acid molecules

Representative for the purpose of the invention, but not limited, methods of Molecular Combing are described by reference to Bensimon, et al., U.S. Pat. No. 6,303,296. These include a process for aligning a nucleic acid on a surface S of a support, wherein the process comprises (a) providing a support having a surface S; (b) contacting the surface S with the nucleic acid; (c) anchoring the nucleic acid to the surface S; (d) contacting the surface S with a first solvent A; (e) contacting the first solvent A with a medium B to form an A/B interface, wherein said medium B is a gas or a second solvent; (f) forming a triple line S/A/B (meniscus) resulting from the contact between the first solvent A, the surface S, and the medium B; and (g) moving the meniscus to align the nucleic acid on the surface.

In this molecular combing process according to or based on the elements and steps described by U.S. Pat. No. 6,303,296, the movement of the meniscus may be achieved by evaporation of the solvent A, which may constitute water or another aqueous medium which may contain surfactants. In this process movement of the meniscus may be achieved by movement of the A/B interface relative to the surface S, wherein S, A and B form a triple line S/A/B constituting the meniscus between the surface S, the solvent A and a medium B which may be a gas (in general air) or another solvent, one example is a water/air meniscus. In this process the surface S may be removed from the solvent A or the solvent A is removed from the surface S in order to move the meniscus. The surface, S, in this process may comprise an organic polymer, an inorganic polymer, a metal, a metal oxide, a sulfide, a semiconductor element, or a combination thereof, for example, it may comprise glass, surface-oxidized silicon, gold, graphite, molybdenum sulfide, or mica. A support useful in this process may comprise a plate, a bead, a fiber, or a particle. In some embodiments, the solvent A is placed between the support of surface S and a second support. Anchoring of nucleic acid(s) in the process may occur via a physicochemical interaction. In some embodiments, the surface S of the support comprises an exposed reactive group having an affinity for the nucleic acid or a molecule with biological activity capable of recognizing the nucleic acid, in other embodiments the surface comprises vinyl, amine, carboxyl, aldehyde, or hydroxyl groups.

The surface S of the support may comprise a substantially monomolecular layer of an organic compound having at least: (a) an attachment group having an affinity for the support; and (b) an exposed group having no or little affinity for the support and the attachment group under attachment conditions, but having an affinity for the nucleic acid or the molecule with biological activity. Anchoring of nucleic acid(s) to the surface may comprise (a) contacting the nucleic acid with the exposed reactive group; (b) adsorbing the nucleic acid to the exposed reactive group at predetermined pH values or ionic content, or by applying an electric voltage, wherein the pH conditions are between a pH resulting in a state of complete adsorption and a pH resulting in an absence of adsorption.

An exposed reactive group may be an ethylenic double bond or an amine group, such as a vinyl or amine group. In some embodiments, adsorption of the nucleic acid may occur at an end of the nucleic acid, the exposed reactive group may be an ethylenic double bond, and the pH is less than 8, preferably between 5 and 6. In another embodiment, the adsorption of the nucleic acid occurs at an end of the nucleic acid, the surface is a polylysine or a silane group, and the exposed group is an amine group. In another embodiment, the adsorption of the nucleic acid occurs at an end of the nucleic acid, the exposed reactive group is an amine group, and the pH is between 9 and 10.

The molecular combing process according to or based on the elements and steps described by U.S. Pat. No. 6,303,296, may be used to detect a nucleic acid in a sample. Such a nucleic acid detection process may comprise (a) providing a support having a surface S; (b) contacting the surface S with a nucleic acid; (c) anchoring the nucleic acid to the surface S; (d) contacting the surface S with a first solvent A; (e) contacting the first solvent A with a medium B, to form an A/B interface, wherein said medium B is a gas or a second solvent; (f) forming a triple line S/A/B (meniscus) resulting from the contact between the first solvent A, the surface S, and the medium B; (g) moving the meniscus to align the nucleic acid on the surface; and (h) detecting, either directly or indirectly, the aligned nucleic acid.

In certain embodiments of the molecular combing processes described by or based on those described by U.S. Pat. No. 6,303,296, the nucleic acid has a sequence complementary to a second nucleic acid sequence in a sample; a molecule with biological activity is biotin, avidin, streptavidin, derivatives thereof, or an antigen-antibody system; the surface exhibits low fluorescence and the nucleic acid is detected, either directly or indirectly, using a fluorescent reagent; the detection is performed using beads; the detection is performed using optical or near field microscopy; or the process may further comprise binding a second molecule to the nucleic acid attached to the surface S, and disrupting nonspecific binding.

Other embodiments of the processes disclosed by U.S. Pat. No. 6,303,296 include a process for detecting a nucleic acid in a sample, wherein the process comprises: (a) providing a support having a surface S; (b) anchoring a second nucleic acid to the surface S; (c) contacting the surface S with a sample A, the sample A comprising a nucleic acid that binds to the second nucleic acid anchored to the surface in a first solvent; (d) binding the nucleic acid in the sample to the anchored nucleic acid; (e) contacting the sample A with a medium B to form an A/B interface, wherein said medium B is a gas or a second solvent; (f) forming a triple line S/A/B (meniscus) resulting from the contact between the sample A, the surface S, and the medium B; (g) moving the meniscus to align the bound nucleic acids on the surface; and (h) detecting, either directly or indirectly, the aligned nucleic acids.

In the molecular combing processes described by or based on those in U.S. Pat. No. 6,303,296, the method of detecting can be ELISA or FISH; or the nucleic acid in the sample is the product of an enzymatic amplification.

The molecular combing procedures described by or based on those described by U.S. Pat. No. 6,303,296, may be used to map genomes or genes that have been modified or repaired, for example, by (a) providing a support having a surface S; (b) contacting the surface S with a nucleic acid to be mapped; (c) anchoring the nucleic acid to the surface S; (d) aligning the anchored nucleic acid on the surface as described above; (e) hybridizing a second nucleic acid of known sequence to the first nucleic acid; and (f) detecting the hybridization between the first nucleic acid and the second nucleic acid. In such processes, the first or the second nucleic acid may comprise genomic DNA; the position and/or the size of the second nucleic acid, which is bound to the first nucleic acid, can be measured; step (d) may comprise stretching the anchored nucleic acid; and the presence or absence of hybridization provides a diagnosis of a pathology or an indication that a genetic modification has been made or a genetic correction made.

Other representative, but not limiting, molecular combing procedures are described by reference to Lebofsky, et al., in WO2008028931, which is incorporated by reference. These methods include a method of detection of the presence of at least one domain of interest on a macromolecule to test, wherein said method comprises the following steps: a) determining beforehand at least two target regions on the domain of interest, designing and obtaining corresponding labeled probes of each target region, named set of probe of the domain of interest, the position of these probes one compared to the others being chosen and forming the specific signature of said domain of interest on the macromolecule to test; b) after spreading of the macromolecule to test on which the probes obtained in step a) are bound, detection of the position one compared to the others of the probes bound on the linearized macromolecule, the detection of the signature of a domain of interest indicating the presence of said domain of interest on the macromolecule to test, and conversely the absence of detection of signature or part of signature of a domain of interest indicating the absence of said domain or part of said domain of interest on the macromolecule to test. The method described above, can be used for determination of the presence of at least two domains of interest and also comprise in step a) determining beforehand at least three target regions on each of the domains of interest. In this method the signature of a domain of interest may result from the succession of spacing between consecutive probes; the position of the domain of interest can be used as reference to locate a chemical or a biochemical reaction; the position of the domain of interest may be used to establish a physical map in the macromolecule encompassing the target region; the domain of interest may consist in a succession of different labelled probes; or some of the probe of the target region may also be part of the signature of at least one other the domain of interest located near on the macromolecule. In this method, all the probes may be labeled with the same label; the probes may be labeled with at least two different labels; the signature of a domain of interest may result of the succession of labels. In this method, the macromolecule may be a nucleic acid, particularly DNA, more particularly double strand DNA; the probes used may be oligonucleotides of at least 1 kb, the spreading of the macromolecule may take place by linearization which may occur before or after binding of the probes on the macromolecules. Linearization of the macromolecule can be made by molecular combing or Fiber Fish. In some embodiments, the binding of at least three probes corresponding to a domain of interest on the macromolecule forms a sequence of at least two spaces chosen between a group of at least two different spaces (for example “short” and “large”), said group being identical for each domain of interest may take place; and the set of probes may comprise in addition two probes (probe 1 or probe 2), each probe capable of binding on a different extremity of the domain of interest, the reading of the signal of one of said probe 1 or probe 2 associated with its consecutive probe in the domain of interest, named “extremity probe couple of start or end” allowing to obtain an information of start or end of reading. In some embodiments, information of start of reading results of the reading of the spacing between the two consecutives probes of the extremity probe couple of start; information of end of reading results of the reading of the spacing between the two consecutives probes of the extremity probe couple of end; or information of start of reading results of the reading of the spacing between the two consecutives probes of the extremity probe couple of start and the information of end of reading results of the reading of the spacing between the two consecutives probes of the extremity probe couple of end, said spacing being different for the extremity probe couple of start and the extremity probe couple of end in order to differentiate information of start and end. In other embodiments of this method, the probes are labeled with fluorescent label or a radioactive label. In some embodiments, the signature comprises a space between the first and the second probe in a set of probes, the space being different from all other spaces in the signature and the space can be used to obtain information about the start of the signature; or the signature comprises a space between the next to last and the last probe in a set of probes, the space being different from all other spaces in the signature and the space can be used to obtain information about the end of the signature.

Specific, but not limited, embodiments of the invention include:

Embodiment 1. A method for detecting, characterizing, quantifying, or determining the efficiency of a gene or genome editing procedure or event comprising performing a genome or gene editing method on target nucleic acid(s) and detecting genetic modifications such as deletion, duplication, amplification, translocation, insertion or inversion using molecular combing or quantifying the efficiency of the genome or gene editing method using molecular combing. The methods described herein may also be used for detecting, characterizing, quantifying, or determining the efficiency of modification or edits or made to other polynucleotides, for example, to segments of a genome outside of a coding or genetic sequence.

Embodiment 2. The method of embodiment 1, wherein the gene or genome editing procedure comprises non-homologous end-joining (NHEJ).

Embodiment 3. The method of embodiment 1 or any one or more of the preceding embodiments, wherein the gene or genome editing procedure comprises homologous recombination comprising at least one of allelic homologous recombination, gene conversion, non-allelic homologous recombination (NAHR), break-induced replication (BIR), single strand annealing (SSA), or other homologous recombination method.

Embodiment 4. The method of embodiment 1 or any one or more of the preceding embodiments, wherein the gene or genome editing procedure comprises activation of endogenous cellular repair machinery and contact of target nucleic acid(s) with a zinc finger nuclease.

Embodiment 5. The method of embodiment 1 or any one or more of the preceding embodiments, wherein the gene or genome editing procedure comprises activation of endogenous cellular repair machinery and contact of target nucleic acid(s) with at least one TALEN (Transcription activator-like effector nuclease).

Embodiment 6. The method of embodiment 1 or any one or more of the preceding embodiments, wherein the gene or genome editing procedure comprises activation of endogenous cellular repair machinery and contact of target nucleic acid(s) with at least one meganuclease. Embodiment 7. The method of embodiment 1 or any one or more of the preceding embodiments, wherein the gene or genome editing procedure comprises activation of endogenous cellular repair machinery and contact of target nucleic acid(s) with at least one meganuclease of the LAGLIDADG (SEQ. ID NO: 1) family.

LAGLIDADG (SEQ. ID NO: 1):

Every polypeptide has 1 or 2 LAGLIDADG (SEQ. ID NO: 1) motifs. The sequence LAGLIDADG (SEQ. ID NO: 1) is a conserved sequence of amino acids where each letter is a code that identifies a specific residue. This sequence is directly involved in the DNA cutting process. Those enzymes that have only one motif work as homodimers, creating a saddle that interacts with the major groove of each DNA half-site. The LAGLIDADG (SEQ. ID NO: 1) motifs contribute amino acid residues to both the protein-protein interface between protein domains or subunits, and to the enzyme's active sites. Enzymes that possess two motifs in a single protein chain act as monomers, creating the saddle in a similar way; see Jurica M S, Monnat R J, Stoddard B L (October 1998). “DNA recognition and cleavage by the LAGLIDADG (SEQ. ID NO: 1) homing endonuclease I-CreI”, Mol. Cell. 2 (4): 469-76 which is incorporated by reference.

Embodiment 8. The method of embodiment 1 or any one or more of the preceding embodiments, wherein the gene or genome editing procedure comprises activation of endogenous cellular repair machinery and contact of target nucleic acid(s) with at least one meganuclease selected from HNH, His-Cys box, GIY-YIG, PD-(D/E)xk and Vsr-like families. Meganucleases described by the embodiments above are described by Belfort M, Roberts R J (September 1995). “Homing endonucleases: keeping the house in order”. Nucleic Acids Res. 25 (17): 3379-88, which is incorporated by reference, describes several structural motifs. Such nucleases may be used for genome, gene and polynucleotide editing steps.

GIY-YIG:

These have only one GIY-YIG motif, in the N-terminal region, that interacts with the DNA in the cutting site. The prototypic enzyme of this family is I-TevI which acts as a monomer. Separate structural studies have been reported of the DNA-binding and catalytic domains of I-TevI, the former bound to its DNA target and the latter in the absence of DNA, see Van Roey, P.; Fox, K M; et al. (July 2001). “Intertwined structure of the DNA-binding domain of intron endonuclease I-TevI with its substrate”. EMBO J. 20 (14): 3631-3637 and Van Roey, P.; Kowalski, Joseph C.; et al. (July 2002). “Catalytic domain structure and hypothesis for function of GIY-YIG intron endonuclease I-TevI”. Nature Structural Biology. 9 (11): 806-811, which are incorporated by reference.

His-Cys Box:

These enzymes possess a region of 30 amino acids that includes 5 conserved residues: two histidines and three cysteines. They co-ordinate the metal cation needed for catalysis. I-PpoI is the best characterized enzyme of this family and acts as a homodimer. Its structure was reported in 1998, see Flick, K.; et al. (July 1998). “DNA binding and cleavage by the nuclear intron-encoded homing endonuclease I-PpoI”. Nature. 394 (6688): 96-101, which is incorporated by reference.

H-N-H:

These have a consensus sequence of approximately 30 amino acids. It includes two pairs of conserved histidines and one asparagine that create a zinc finger domain. I-HmuI is the best characterized enzyme of this family, and acts as a monomer. Its structure was reported in 2004, see Shen, B. W.; et al. (September 2004). “DNA binding and cleavage by the HNH homing endonuclease I-HmuI”. J. Mol. Biol. 342 (1): 43-56, which is incorporated by reference.

PD-(D/E)xK:

These enzymes contain a canonical nuclease catalytic domain typically found in type II restriction endonucleases. The best characterized enzyme in this family, I-Ssp6803I, acts as a tetramer. Its structure was reported in 2007, see Zhao, L.; et al. (May 2007). “The restriction fold turns to the dark side: a bacterial homing endonuclease with a PD-(D/E)-XK motif”. EMBO Journal. 26 (9): 2432-2442, which is incorporated by reference.

Vsr-Like:

These enzymes were discovered in the Global Ocean Sampling Metagenomic Database and first described in 2009. The term ‘Vsr-like’ refers to the presence of a C-terminal nuclease domain that displays recognizable homology to bacterial Very Short Patch Repair (Vsr) endonucleases, see Dassa, B.; et al. (March 2009). “Fractured genes: a novel genomic arrangement involving new split inteins and a new homing endonuclease family”. Nucleic Acids Research. 37 (8): 2560-2573, which is incorporated by reference.

Embodiment 9. The method of embodiment 1, wherein the gene or genome editing procedure comprises activation of endogenous cellular repair machinery and contact of target nucleic acid(s) with at least one I-CreI or I-SceI meganuclease.

Embodiment 10. The method of embodiment 1 or any one or more of the preceding embodiments, wherein the gene or genome editing procedure comprises activation of endogenous cellular repair machinery and contact of target nucleic acid(s) with a CRISPR/Cas9 system or CRISPR/Cas9 variant system.

Embodiment 11. The method of embodiment 1 or any one or more of the preceding embodiments, wherein the gene or genome editing procedure comprises activation of endogenous cellular repair machinery and contact of target nucleic acid(s) with a type I CRISPR/Cas9 system.

Embodiment 12. The method of embodiment 1 or any one or more of the preceding embodiments, wherein the gene or genome editing procedure comprises activation of endogenous cellular repair machinery and contact of target nucleic acid(s) with a type II CRISPR/Cas9 system.

Embodiment 13. The method of embodiment 1 or any one or more of the preceding embodiments, wherein the gene or genome editing procedure comprises activation of endogenous cellular repair machinery and contact of target nucleic acid(s) with a type III CRISPR/Cas9 system.

Embodiment 14. The method of embodiment 1 or any one or more of the preceding embodiments, wherein the gene or genome editing procedure comprises activation of endogenous cellular repair machinery and contact of target nucleic acid(s) with a type IV CRISPR/Cas9 system.

Embodiment 15. The method of embodiment 1 or any one or more of the preceding embodiments, wherein the gene or genome editing procedure comprises activation of endogenous cellular repair machinery and contact of target nucleic acid(s) with a type V CRISPR/Cas9 system.

Embodiment 16. The method of embodiment 1 or any one or more of the preceding embodiments, wherein the gene or genome editing procedure comprises activation of endogenous cellular repair machinery and contact of target nucleic acid(s) with a type VI CRISPR/Cas9 system.

Embodiment 17. The method of embodiment 1 or any one or more of the preceding embodiments, wherein the gene or genome editing procedure produces a nucleic acid rearrangement comprising a gene knockout.

Embodiment 18. The method of embodiment 1 or any one or more of the preceding embodiments, wherein the gene or genome editing procedure produces a nucleic acid rearrangement comprising a mutation other than a single nucleotide variation.

Embodiment 19. The method of embodiment 1 or any one or more of the preceding embodiments, wherein the gene or genome editing procedure produces a nucleic acid rearrangement comprising a correction. Such a correction may comprise a correction to a coding sequence, a correction in a genetic sequence outside of the coding region or a correction outside of a gene region.

Embodiment 20. The method of embodiment 1 or any one or more of the preceding embodiments, wherein the gene or genome editing procedure produces a nucleic acid rearrangement comprising a deletion. Such a deletion may comprise a deletion to a coding sequence, a deletion in a genetic sequence outside of the coding region or a deletion outside of a gene region.

Embodiment 21. The method of embodiment 1 or any one or more of the preceding embodiments, wherein the gene or genome editing procedure produces a nucleic acid rearrangement comprising an insertion. Such an insertion may comprise an insertion into a coding sequence, an insertion into a genetic sequence outside of the coding region or an insertion outside of a gene region.

Embodiment 22. The method of embodiment 1 or any one or more of the preceding embodiments, wherein the gene or genome editing procedure produces a nucleic acid rearrangement comprising a duplication. Such a duplication may comprise a duplication to a coding sequence, a duplication in a genetic sequence outside of the coding region or a duplication outside of a gene region.

Embodiment 23. The method of embodiment 1 or any one or more of the preceding embodiments, wherein the gene or genome editing procedure produces a nucleic acid rearrangement comprising an amplification. Such an amplification may comprise an amplification to a coding sequence, an amplification in a genetic sequence outside of the coding region or an amplification outside of a gene region.

Embodiment 24. The method of embodiment 1 or any one or more of the preceding embodiments, wherein the gene or genome editing procedure produces a nucleic acid rearrangement comprising a translocation. Such a translocation may comprise a translocation to a coding sequence, a translocation in a genetic sequence outside of the coding region or a translocation outside of a gene region.

Embodiment 25. The method of embodiment 1 or any one or more of the preceding embodiments, wherein the gene or genome editing procedure produces a nucleic acid rearrangement comprising an inversion. Such an inversion may comprise an inversion to a coding sequence, an inversion in a genetic sequence outside of the coding region or an inversion outside of a gene region.

Embodiment 26. The method of embodiment 1 or any one or more of the preceding embodiments that detects or quantifies a nucleic acid rearrangement or the lack of a nucleic acid rearrangement or off-target events with at least 5, 10, 20, 30, 40, 50, 60, 70, 80, 90, or 100%, accuracy or efficiency.

Embodiment 27. The method of any of the preceding embodiments that detects or quantifies a nucleic acid rearrangement or the lack of a nucleic acid rearrangement or off-target events with at least 5, 10, 20, 30, 40, 50, 60, 70, 80, 90, 100% or more accuracy or efficiency (where 100% indicates double the accuracy or efficiency of a comparative conventional method) than at least one conventional method of restriction site selection, PAGE-based genotyping method, enzymatic mismatch cleavage-based assays, subcloning a target region, subcloning of the targeted region, high-resolution melting curve (HRM) analysis, next gene sequencing, or droplet digital PCR or any other conventional methods that detect or quantify rearrangements.

Embodiment 28. The method of embodiment 1 or any one or more of the preceding embodiments, wherein the genome or gene editing procedure or event occurs in vivo or in a sample obtained from in vivo, optionally after treatment of a subject with a polynucleotide, drug, radiation, immunological agent or other therapy.

Embodiment 29. The method of embodiment 1 or any one or more of the preceding embodiments, further comprising detecting a polynucleotide comprising a genomic or gene rearrangement, deletion, duplication, amplification, translocation, insertion or inversion or selecting a sample comprising said polynucleotide.

Embodiment 30. A rearranged or edited polynucleotide selected or otherwise identified or validated by the method of embodiment 1 or any one or more of the preceding embodiments.

Embodiment 31. The rearranged or edited polynucleotide of embodiment 30 that is cDNA or DNA.

Embodiment 32. Use of a polynucleotide, drug, radiation, immunological agent or other therapeutic agent in combination with one or more genome or gene editing or molecular combing agents described by embodiment 1 or any one or more of the preceding embodiments for treatment of the human or animal body, for example, by genetic surgery or therapy, and/or for diagnosis thereof.

Embodiment 33. A method for controlling quality of a polynucleotide, genome or gene editing procedure that uses at least one modified nuclease comprising:
(i) editing one or more polynucleotide(s) of interest using at least one modified nuclease,
(ii) detecting, characterizing or quantifying the edited polynucleotide(s) by contacting them with fluorescent polynucleotide(s) that hybridize to them and performing molecular combing, and
(iii) comparing the edited polynucleotides hybridized to said fluorescent polynucleotides of interest to one or more control polynucleotides, which have not been treated with the modified nuclease, hybridized to said fluorescent polynucleotide(s), thus determining the efficiency, accuracy or specificity of the polynucleotide editing procedure using the modified nuclease;
(iv) optionally, selecting a modified nuclease based polynucleotide, genome or gene editing procedure that is most accurate or efficient for correction or modification of a particular polynucleotide, gene or genome or for a therapeutic application. The editing procedure may be performed with any of the modified nucleases described herein or two or more of such nucleases, for example, when different parts of a polynucleotide, gene or genome are to be modified. This procedure may be performed using molecular combing methods known in the art or those described herein.

Embodiment 34. The method according to embodiment 1 or one or more of the preceding embodiments, wherein said performing a genome or gene editing method comprises:

a first step of contacting the modified nucleic acid sequence with the corresponding labeled standard reference genetic sequence of interest, said genetic modifications, deletions or replacement in the genomic DNA having been operated with an engineered nuclease or meganuclease,

a second step of comparing said modified nucleic acid sequence with the corresponding standard reference nucleic acid sequence of interest.

Embodiment 35. A method according to embodiment 1 or one or more of the preceding embodiments comprising a step of quantification of the number of deletions events or of unwanted genetic events or of unexpected rearrangements occurred and simultaneously the identification of the genetic modifications or of the deletion in the targeted region of the modified genome.

Embodiment 36. A method according to embodiment 1 or one or more of the preceding embodiments comprising:

a first step a step of quantification of the number of deletions events or of unwanted genetic events or of unexpected rearrangements occurred and said step being followed by a second step allowing the identification of the deletion and then the quantification of unexpected rearrangements or unwanted genetic events in the targeted region or sequence of the modified genome wherein the said modifications are operated by engineered nucleases or mega nucleases,

or optionally followed by a second step allowing the identification of the deletion and then the quantification of unexpected rearrangements or unwanted genetic events in the targeted region or sequence of the modified genome wherein the said modifications are operated by engineered nucleases or mega nucleases.

Embodiment 37. The method according to embodiment 1 or one or more of the preceding embodiments, wherein the modified nucleic acid is genomic DNA or a recombinant or synthetic DNA hybridizing under stringent conditions with the reference or normal wild type of DNA.

Embodiment 38. The method according to Embodiment 1 or one or more of the preceding embodiments, wherein said detecting or quantifying DNA modifications comprises the quantifying the number of deletions events in the BRCA1 genomic DNA and identifying the said genetic modifications in the targeted cellular genomic DNA.

Embodiment 39. A method for detecting, characterizing, quantifying, or determining the efficiency of, a gene or genome editing procedure or event comprising:

editing a target nucleic acid(s) in a gene or genome and

detecting or quantifying at least one genetic modification, deletion, duplication, amplification, translocation, insertion or inversion in the edited target nucleic acid using molecular combing.

Embodiment 40. The method of embodiment 39, wherein the editing comprises non-homologous end-joining (NHEJ) in a double strand break in the target nucleic acid(s).

Embodiment 41. The method of embodiment 39 or of any one or more of the preceding embodiments, wherein the editing comprises homologous recombination in the target nucleic acid(s) comprising at least one of allelic homologous recombination, gene conversion, non-allelic homologous recombination (NAHR), break-induced replication (BIR), or single strand annealing (SSA).

Embodiment 42. The method of embodiment 39 or of any one or more of the preceding embodiments, wherein the editing procedure comprises activating endogenous cellular repair machinery and contacting the target nucleic acid with a zinc finger nuclease.

Embodiment 43. The method of embodiment 39 or of any one or more of the preceding embodiments, wherein the editing comprises activation of endogenous cellular repair machinery and contacting the target nucleic acid(s) with at least one TALEN (Transcription activator-like effector nuclease).

Embodiment 44. The method of embodiment 39 or of any one or more of the preceding embodiments, wherein the editing comprises activating endogenous cellular repair machinery and contacting the target nucleic acid(s) with at least one meganuclease.

Embodiment 45. The method of embodiment 39 or of any one or more of the preceding embodiments, wherein the editing comprises activating endogenous cellular repair machinery and contacting the target nucleic acid(s) with at least one meganuclease of the LAGLIDADG (SEQ. ID NO: 1) family.

Embodiment 46. The method of embodiment 39 or of any one or more of the preceding embodiments, wherein the editing comprises activating endogenous cellular repair machinery and contacting the target nucleic acid(s) with at least one I-CreI or I-SceI meganuclease.

Embodiment 47. The method of embodiment 39 or of any one or more of the preceding embodiments, wherein the editing comprises activating endogenous cellular repair machinery and contacting the target nucleic acid(s) with a CRISPR/Cas9 system or CRISPR/Cas9 variant system.

Embodiment 48. The method of embodiment 39 or of any one or more of the preceding embodiments,

wherein the editing comprises activating endogenous cellular repair machinery and contacting the target nucleic acid(s) with a type I CRISPR/Cas9 system;

wherein the editing comprises activating endogenous cellular repair machinery and contacting the target nucleic acid(s) with a type II CRISPR/Cas9 system;

wherein the editing comprises activating endogenous cellular repair machinery and contacting the target nucleic acid(s) with a type III CRISPR/Cas9 system;

wherein the editing comprises activation of endogenous cellular repair machinery and contact of target nucleic acid(s) with a type IV CRISPR/Cas9 system;

wherein the editing comprises activating endogenous cellular repair machinery and contacting the target nucleic acid(s) with a type V CRISPR/Cas9 system; or

wherein the editing comprises activating endogenous cellular repair machinery and contacting the target nucleic acid(s) with a type VI CRISPR/Cas9 system.

Embodiment 49. The method of embodiment 39 or of any one or more of the preceding embodiments, wherein the editing produces a nucleic acid rearrangement that knocks out a gene.

Embodiment 50. The method of embodiment 39 or of any one or more of the preceding embodiments,

wherein the editing produces a nucleic acid rearrangement that mutates the target nucleic acid(s);

wherein the editing produces a nucleic acid rearrangement comprising a gene correction;

wherein the editing produces a nucleic acid rearrangement comprising a deletion;

wherein the editing produces a nucleic acid rearrangement comprising an insertion;

wherein the editing produces a nucleic acid rearrangement comprising a duplication;

wherein the editing produces a nucleic acid rearrangement comprising an amplification;

wherein the editing produces a nucleic acid rearrangement comprising a translocation; or

wherein the editing produces a nucleic acid rearrangement comprising an inversion.

Embodiment 51. The method of embodiment 39 or of any one or more of the preceding embodiments that quantifies a number of the nucleic acid rearrangements produced by the editing of the target nucleic acid(s).

Embodiment 52. The method of embodiment 39 or of any one or more of the preceding embodiments that quantifies a number of the nucleic acid rearrangements produced by the editing of the target nucleic acid(s) faster or with a higher degree of accuracy than a conventional quantification method selected from the group consisting of restriction site selection, PAGE-based genotyping assay, enzymatic mismatch cleavage-based assay, subcloning a target region, high-resolution melting curve (HRM) analysis, Next-Gen gene sequencing, and droplet digital PCR.

Embodiment 53. The method of embodiment 39 or of any one or more of the preceding embodiments, wherein the editing occurs in vivo or ex vivo, optionally after treatment of a subject with a polynucleotide, drug, radiation, immunological agent or other therapy.

Embodiment 54. The method according to embodiment 39 or any one or more of the preceding embodiments, wherein said editing comprises:

contacting the target nucleic acid that has been edited with an engineered nuclease or meganuclease(s) with an unedited control target sequence, and

comparing said edited target nucleic acid sequence with the sequence of the unedited control target sequence.

Embodiment 55. The method according to embodiment 39 or any one or more of the preceding embodiments, wherein a number of deletions or other unwanted or unexpected genetic events in the target nucleic acid(s) as well as the number of desired edits to the target nucleic acid(s) are quantified by molecular combing.

Embodiment 56. The method of embodiment 54, wherein the editing is performed using an engineered nuclease or meganuclease

Embodiment 57. The method according to embodiment 39 or of any one or more of the preceding embodiments, wherein said target nucleic acid(s) comprise BRCA1 genomic DNA.

Embodiment 58. The method of embodiment 39 or of any one or more of the preceding embodiments, wherein the genome or gene editing procedure or event occurs in vivo or in a sample obtained from in vivo, optionally after treatment of a subject by gene therapy or with a polynucleotide, drug, radiation, immunological agent or other therapy.

Embodiment 59. A method for determining the efficiency, accuracy or specificity of a polynucleotide editing procedure that uses at least one modified nuclease comprising:
(i) editing one or more polynucleotide(s) of interest using at least one modified nuclease,
(ii) contacting the edited polynucleotide(s) with labelled polynucleotide(s) that hybridize to them and performing molecular combing of the fluorescent labeled polynucleotides, and
(iii) comparing the edited polynucleotides hybridized to said labelled polynucleotides to one or more control polynucleotides, which have not been treated with the modified nuclease, hybridized to said labelled polynucleotide(s), thus determining the efficiency, accuracy or specificity of the polynucleotide editing procedure using the modified nuclease; and
(iv) optionally, selecting a modified nuclease based polynucleotide editing procedure that is most accurate or efficient for correction or modification of a particular polynucleotide of interest.

Embodiment 60. The method according to any one of Embodiments 1 or 29 or 59, wherein target nucleic acid(s) or the target polynucleotide of interest comprises BRCA1 genomic DNA.

Embodiment 61. A method according to any one of Embodiments 1 to 60 that comprises the following steps:
(a) preparing embedded DNA material from the assessed sample comprising genome or genetic material, such as embedded DNA agarose plugs;
(b) extracting the embedded DNA material recovered from step (a) to recover DNA and performing Molecular Combing on the extracted DNA by stretching DNA and recovering immobilized linear and parallel strands of nucleic acid; wherein the extraction step optionally encompass a step of digesting the embedded DNA material with proteinase;
(c) on combed DNA, hybridizing labelled probes wherein said probes are specific for the detection of the gene or genome editing events
(d) detecting combed DNA hybridized with probes
(e) detecting and/or quantitating the editing events by discriminating between intact DNA molecules and edited DNA molecules,
wherein before step (a) and/or between steps (a) and (b) a step of treating the assessed sample or the genome or the genetic material of said sample with editing procedure, in particular with a meganuclease is performed and optionally,
wherein a control sample is treated with steps (a) to (e) but does not undergo the editing procedure, for comparison with the assessed sample.

The following Examples illustrate particular non-limited embodiments or aspects of the invention or support therefore.

### EXAMPLES

### Example 1—Detection of Genome Editing Events Induced by Meganucleases

Preparation of Embedded DNA Plugs from Viral Particles

Agarose plugs containing the recombinant HSV-1 (rHSV-1) (Grosse, Huot et al. 2011) were prepared with modified procedure as described in Mahiet et al. (Mahiet, Ergani et al. 2012) and in WO 2011/132078 (EP 2 561 104 B1). Briefly, rHSV-1 particles were resuspended in 1×PBS at a concentration of 5·10⁶ viral particles/mL, and mixed thoroughly at a 1:1 ratio with a 1.2% w/v solution of low-melting point agarose (Nusieve GTG, ref. 50081, Cambrex) prepared in PBS, at 50° C. 904, of the viral particles/agarose mix was poured in a plug-forming well (BioRad, ref. 170-3713) and left to cool at least 30 min at 4° C. Embedded recombinant viral particles were lysed in 0.1% SDS—0.5M EDTA (pH8.0) solution at 50° C. for 30 minutes. After three washing steps in 0.5M EDTA (pH 8.0) buffer of 10 minutes at room temperature, plugs were digested by overnight incubation at 50° C. with 2 mg/mL Proteinase K (Eurobio code GEXPRK01, France) in 250 μL digestion buffer (0.5M EDTA (pH8.0).

In Vitro I-SceI-Induced Double Strand Breaks

First, agarose plugs of embedded DNA from recombinant viral particles are incubated in 100 μl 1× Tango Buffer without Mg-Acetate (New England Biolabs) diluted in TE 10:1 with 20 u of I-SceI for 2 h on ice. H₂O replaced I-SceI in the untreated-ISceI samples used as negative control. Then, Mg-Acetate is added to a final concentration of 10 μM to allow I-SceI activity starting and incubated for 2 h at 37° C. After three washing steps in TEN 10:20:100 of 30 minutes at room temperature, plugs were again digested by overnight incubation at 50° C. with 2 mg/mL Proteinase K (Eurobio code GEXPRK01, France) in 250 μL digestion buffer (0.5M EDTA (pH8.0).

DNA Extraction and Molecular Combing

Agarose plugs of embedded DNA from I-SceI-untreated and I-SceI-treated rHSV-1 were treated for combing DNA as previously described (Schurra and Bensimon 2009). Briefly, plugs were first washed 3 times in 15 ml TE 10:1 for 30 min and then melted at 68° C. in a IVIES 0.5 M (pH 5.5) solution for 20 min, and 1.5 units of beta-agarase (New England Biolabs, ref. M0392S, MA, USA) was added and left to incubate for up to 16 h at 42° C. The DNA solution was then poured in a Teflon reservoir and Molecular Combing was performed using the Molecular Combing System (Genomic Vision S.A., Paris, France) and Molecular Combing coverslips (20 mm×20 mm, Genomic Vision S.A., Paris, France). The combed surfaces were dried for 4 hours at 60° C.

Labelling of HSV-1 Probes

The 41 HSV-1 probes and the LacZ probe (containing the I-SceI site) are as described in Mahiet et al. (Mahiet, Ergani et al. 2012) and in WO 2011/132078 (EP 2 561 104 B1). Briefly, the labelling of the probes was performed using conventional random priming protocols. For the HSV-1 probes, the BioPrime® DNA kit (Invitrogen, code: 18094-011, CA, USA) was used with biotin-11-dCTP according to the manufacturer's instructions, except the labelling reaction was allowed to proceed overnight. For efficient labelling, the HSV-1 probes were gathered into groups of 3 to 5 (200 ng of each plasmid). The LacZ probe (200 ng) was labelled with Alexa Fluor® 488-7-OBEA-dCTP. For this labelling, the dNTP mix from the kit was replaced by the mix containing of 40 μM of each dATP, dTTP and dGTP, 20 μM of dCTP and 20 μM of Alexa Fluor 488-7-OBEA-dCTP (ThermoFischer Scientific, ref: C21555). The reaction products were visualized on an agarose gel to verify the synthesis of DNA.

Hybridization of HSV-1 Probes on Combed Viral DNA and Detection

Subsequent steps were also performed essentially as previously described in Schurra and Bensimon (Schurra and Bensimon 2009). Briefly, a mix of labelled probes (250 ng of each probe) were ethanol-precipitated together with 10 μg herring sperm DNA and 2.5 μg Human Cot-1 DNA (Invitrogen, ref. 15279-011, CA, USA), resuspended in 20 μL of hybridization buffer (50% formamide, 2×SSC, 0.5% SDS, 0.5% Sarkosyl, 10 mM NaCl, 30% Block-aid (Invitrogen, ref. B-10710, CA, USA). The probe solution and probes were heat-denatured together on the Hybridizer (Dako, ref. 52451) at 90° C. for 5 min and hybridization was left to proceed on the Hybridizer overnight at 37° C. Slides were washed 3 times in 50 formamide, 2×SSC and 3 times in 2×SSC solutions, for 5 min at room temperature. After the last washing steps, the hybridized coverslips were gradually dehydrated in 70%, 90% and 100% ethanol solution and air dried. Detection of labelled probes was carried out using two or three layers of antibodies in a 1:25 dilution. Biotin-11-dCTP-labelled probes were revealed with an Alexa Fluor® 594 conjugated-streptavidin (Invitrogen), as first layer, followed by an incubation with a biotinylated goat anti-streptavidin antibody (Vector Laboratories) and then of an Alexa Fluor® 594 coupled-streptavidin. Alexa Fluor® 488-7-OBEA-dCTP labelled LacZ probe was consecutively revealed with an Alexa Fluor® 488-conjugated polyclonal rabbit antibody (Invitrogen), then a polyclonal Alexa Fluor® 488-conjugated goat anti-Rabbit antibody (Invitrogen) as final layer. For each layer, 20 μL of the antibody solution was added on the slide and covered with a combed coverslip and the slide was incubated in humid atmosphere at 37° C. for 20 min. The slides were washed 3 times in a 2×SSC, 1% Tween20 solution for 3 min at room temperature between each layer and after the last layer. After the last washing steps, all glass cover slips were dehydrated in ethanol and air dried.

Analysis of HSV-1 Detected Signals

Hybridized-combed DNA from recombinant viral particles were scanned without any mounting medium using an inverted automated epifluorescence microscope, equipped with a 40× objective (ImageXpress Micro, Molecular Devices, USA) and the signals can be detected visually or automatically by an in house software (Gvlab 0.4.2). For quantification of the digestion efficiency, all fluorescent signal arrays with an intact LacZ probe, e.g. an Alexa Fluor 488 fluorescent signal is flanked by Alexa Fluor® 594 signals, are considered as intact rHSV-1 molecules (% ND) whereas the fluorescent signal array with an interrupted LacZ probes, e.g. Alexa Fluor 488 fluorescent signal flanked by a Alexa Fluor® 594 signal at only one of its extremities, are thought to be either rHSV-1 molecules with I-SceI-induced DBS or molecules that have been randomly sheared during the experimental process (% D). The basal level of sheared DNA molecules is evaluated in the control condition in which no I-SceI enzyme was added. In these conditions, the global digestion efficiency is calculated as follows:

$$Global digestion efficiency = \frac{\% Dsample - \% Dcontrol}{\% NDcontrol} \times 100$$

Semi-Quantitative PCR

After Molecular Combing, the DNA solution is transferred in a dialysis tube and the dialysis is performed against 3 liters of TE 10:1 at 4° C. overnight. The semi-quantitative PCR is performed using serial dilution of the DNA solution (1:1 to 1:1000) as template with the different primer pairs (25 μmol each) as described in Table A and the Expand™ High Fidelity PCR System according to the manufacturer's instructions (Roche Diagnostics). The amplification products were visualized on a 2% agarose gel to verify the size of DNA. Since the Sce-1a and Sce-1b primer pairs flanked the I-SceI site, no amplification product is obtained in case of I-SceI-induced DBS whereas the Sce-2 and Sce-3 primer pairs are used as positive control since reaction products are obtained from both intact and I-SceI-induced DBS rHSV-1 DNA molecules.

TABLE A
Primers sequences used for the amplification
of rHSV-1 region by PCR.

| | | Product |
|---|---|---|
| Primer Name | Sequence (5′−>3′) | Size |
| Sce-1a_For | GAA TCC CAG TCC GTC CGA TA | 138 pb |
| | (SEQ. ID NO: 2) | |
| Sce-1_Rev | CGA CGG GAT CTA TCA TCG TT | |
| | (SEQ. ID NO: 3) | |
| Sce-1b_For | TCC GTC CGA TAT TAC CCT GT | 129 pb |
| | (SEQ. ID NO: 4) | |
| Sce-1_Rev | CGA CGG GAT CTA TCA TCG TT | |
| | (SEQ. ID NO: 5) | |
| Sce-2_For | GCT CGG ATC CAC TAG TCC AG | 122 pb |
| | (SEQ. ID NO: 6) | |
| Sce-2_Rev | GTG CTG CAA GGC GAT TAA GT | |
| | (SEQ. ID NO: 7) | |
| Sce-3_For | CAC CAA AAT CAA CGG GAC TT | 136 pb |
| | (SEQ. ID NO: 8) | |
| Sce-3_Rev | AGC CAG TAA GCA GTG GGT TC | |
| | (SEQ ID NO: 9 | |

Detection and Quantification of 1-SceI Meganuclease-Induced DBS in rHSV-1 DNA Molecules

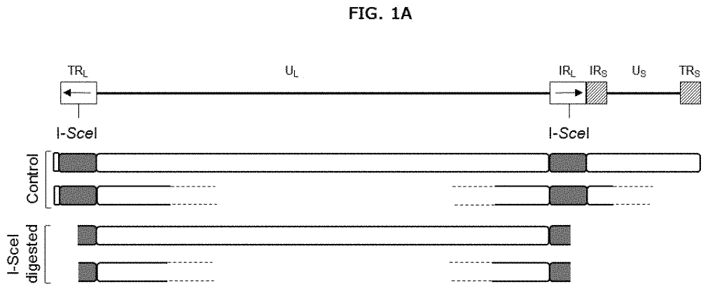

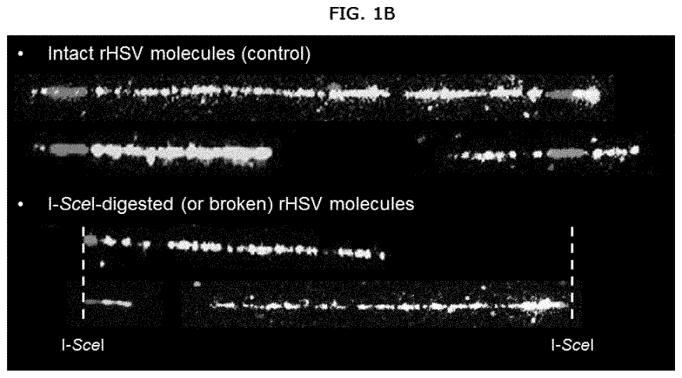

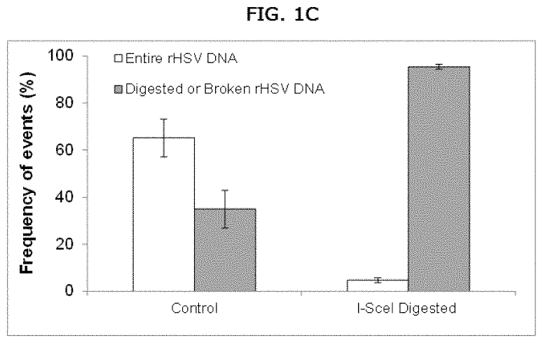

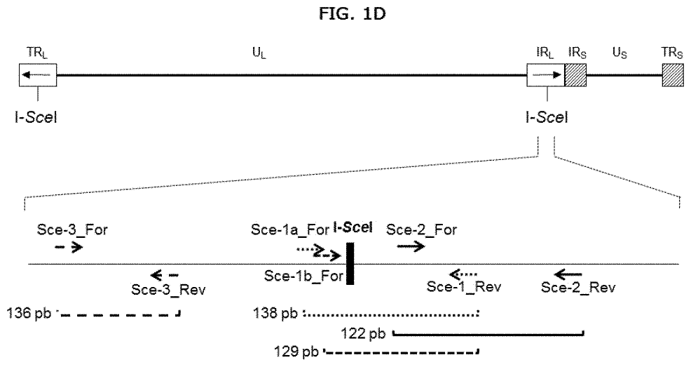

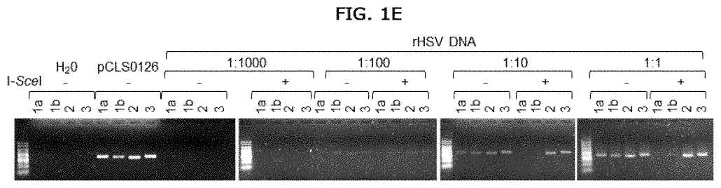

The inventors applied Molecular Combing to uniformly stretch rHSV-1 DNA that has been treated by I-SceI meganuclease in the agarose plugs and hybridized the resulting combed rHSV-1 DNA with labelled adjacent and overlapping DNA probes (FIG. 1A; HSV-1: Alexa Fluor® 594-fluorescence; LacZ: Alexa Fluor® 488-fluorescence) to discriminate between intact rHSV-1DNA molecules and rHSV-1 molecules with ISce-I-induced DBS. 3 independent experiments consisting of a pair of agarose plugs with embedded rHSV-1 DNA that are treated or not by I-SceI meganuclease as described in the “In vitro I-SceI-induced double strand breaks” section. Immunofluorescence microscopy (FIG. 1B) exhibit between 929 and 1473 multicolor linear patterns per conditions (Table B) that fulfilled the criteria for evaluation (see “Analysis of HSV-1 detected signals” section). Classification of the signals between intact rHSV-1 signals and signals with I-SceI-induced DBS showed that the I-SceI activity is almost complete with a mean activity above 90% (Table B and FIG. 1C). To confirm the I-SceI activity observed by Molecular Combing, we conducted a semi-quantitative PCR analysis with different primer pairs as described in Table A and showed in FIG. 1D using control and I-SceI-treated DNA as template. The different PCR tubes are set up such that they either vary in the amount of DNA template (1:1 to 1:1000 serial dilution of control or treated rHSV-1 DNA). This is because PCR amplification, though theoretically logarithmic, is not so at low or high number of amplification cycles. The logarithmic or exponential amplification usually occurs only during the middle cycles, and this depends on the concentration of target template. Comparison can therefore be done only during this phase. After amplification, same volume of reaction products are electrophoresed on a 2% agarose gel. Images of stained PCR products are then obtained and analyzed by visual comparison (FIG. 1E). Absence of PCR products with Sce-1a and Sce-1b primers pairs mean that the I-SceI meganuclease introduced DSB in the rHSV-1 DNA whereas the presence of a PCR product with these primers pairs notified absence or undetectable I-SceI activity. Sce-2 and Sce-3 primer pairs are used as positive control to exclude the degradation of the rHSV-1 DNA thus a PCR product should be observed whatever the conditions (I-SceI-treated or control rHSV-1). As expected, no PCR products were obtained with the negative control (H₂O) whereas a PCR product is amplified with the positive control (pCLS0126) whatever the primer pairs. For each pair of primers, a PCR product is amplified from the rHSV-1 DNA that has not been treated with the I-SceI meganuclease. For the I-SceI-treated samples, a band corresponding to a PCR product with the primer pairs Sce-1a and 1b is observed in non-diluted DNA sample (1:1) but with a weaker intensity compared to the PCR product amplified with the Sce2 and Sce3 primers pairs. In diluted samples (1:10 to 1:100), the amplification product with the primer pairs Sce-1a and 1b is undetectable whereas a PCR product is still observed for the Sce2 and Sce3 primers pairs. These results confirm that the activity of I-SceI meganuclease is almost complete thus confirming the data obtained by Molecular Combing analysis.

These results show that the Molecular Combing techniques of the invention are powerful methods for the detection of meganuclease-induced DSB events at the level of the unique molecule and to quantify its activity efficacy.

TABLE B
Data obtained from 3 independent experiments.

| | | Number of signals | | | |
|---|---|---|---|---|---|
| Experi- | | | I-SceI- | | I-SceI |
| ment | Conditions | Intact | induced DBS | Total | efficacy |
| 1 | Control | 822 | 651 | 1473 | 89.71% |
| | I-SceI-treated | 65 | 1067 | 1132 | |
| 2 | Control | 886 | 394 | 1280 | 94.71% |
| | I-SceI-treated | 34 | 895 | 929 | |
| 3 | Control | 989 | 417 | 1406 | 93.47% |
| | I-SceI-treated | 59 | 1225 | 1284 | |
| | | | | Mean ± SD | 92.63% ± 2.6 |

### Example 2—Detection of Genome Editing Events Induced by CRISPR-Cas9 RNA Guided Nucleases

BRCA Gene Editing in HEK293 Cells

HEK293 cell lines were cultivated in complete DMEM media (DMEM high glucose+10% FBS+/Pen/Strep antibiotics) at 37° C. in 5% CO₂ atmosphere. Cells were maintained by splitting every 4-5 days at a ratio of 1:10.

To create a 6.5 kb deletion in the BRCA gene in HEK293 cells, gRNA pairs were designed (see Table C) and cloned in the pSpCas9(BB)-2A-Puro (PX459) vector (ALSTEM, CA, USA). 3×10⁵ cells were transfected with 1 μg of each BRCA-Left-gRNA and BRCA-Right-gRNA using 6W of NanoFect transfection reagent. Transfection with the different combinations of BRCA-Left-gRNA and BRCA-Right-gRNA was performed. An isogenic cell culture, e.g. HEK293 cells not transfected with the gRNA vectors, was also used as negative control. After 4 days, transfected cells were harvested and the genomic DNA was extracted using Genomic DNA extraction kit (Avegene).

TABLE C
gRNA sequence for BRCA targeting

| | | SEQ | |
|---|---|---|---|
| gRNA Name | Sequence (5′−>3′) | ID NO: | PAM |
| BRCA-Left-gRNA1 | GGGGTGCGGTTTATTCATAC | 10 | AGG |
| BRCA-Left-gRNA4 | CCTGAGGCGGGTGGATCATG | 11 | AGG |
| BRCA-Left-gRNA7 | ATTCATACAGGTAGTGAGAG | 12 | TGG |
| BRCA-Right-gRNA4 | CCACACCACCAATTACCACA | 13 | AGG |
| BRCA-Right-gRNA9 | ATGGGAGAAGGTCATAGATG | 14 | AGG |
| BRCA-Right-gRNA12 | GTGGAGGCAGAGATTACACA | 15 | AGG |

PCR Characterization of the Transfected Cell Pool

The genomic DNA was subsequently used for PCR to amplify the targeted BRCA region using the Phusion® High-Fidelity DNA polymerase and the primers pairs described in Table D. 2% agarose gel to verify the size of DNA. Since the BRCA-Left-PCR-F and BRCA-Left-PCR-R primer pair is used as positive control, amplification reaction is not affected by the CRISPR-Cas9-induced BRCA deletion. For BRCA-Left-PCR-F and BRCA-Right-PCR-R primer pair that flanked the targeted BRCA site, the expected 7224 bp-amplification product cannot be amplified in the isogenic control since the PCR extension time is only 30 s whereas a shorter PCR products (between 490 and 651 bp depending on the gRNA combination, see table E) is obtained in samples with the expected editing events in the BRCA1 gene.

TABLE D
PCR primers and Tm value

| Primer Name | Sequence (5′−>3′) | Tm (° C.) |
|---|---|---|
| BRCA-Left- | TGGCTTCAAAGAGACTGCGA | 66.2 |
| PCR-F | (SEQ ID NO: 16) | |
| BRCA-Left- | TGTCAGCATTTGGCTCCACT | |
| PCR-R | (SEQ. ID NO: 17) | |
| BRCA-Left- | TGGCTTCAAAGAGACTGCGA | 66.2 |
| PCR-F | (SEQ. ID NO: 18) | |
| BRCA-Right- | GGCCAGTGTAGCTGGAGTAATTTG | |
| PCR-R | (SEQ. ID NO: 19) | |

TABLE E
gRNA combinations and their expected PCR size

| Conditions | gRNA pairs | PCR size (bp) |
|---|---|---|
| 1 | BRCA-Left-gRNA1 + BRCA-Right- | 651 |
| | gRNA4 | |
| 7 | BRCA-Left-gRNA1 + BRCA-Right- | 596 |
| | gRNA9 | |
| 8 | BRCA-Left-gRNA1 + BRCA-Right- | 572 |
| | gRNA12 | |
| 4 | BRCA-Left-gRNA4 + BRCA-Right- | 569 |
| | gRNA4 | |
| 9 | BRCA-Left-gRNA4 + BRCA-Right- | 514 |
| | gRNA9 | |
| 5 | BRCA-Left-gRNA4 + BRCA-Right- | 490 |
| | gRNA12 | |
| 6 | BRCA-Left-gRNA7 + BRCA-Right- | 639 |
| | gRNA4 | |
| 3 | BRCA-Left-gRNA7 + BRCA-Right- | 584 |
| | gRNA9 | |
| 2 | BRCA-Left-gRNA7 + BRCA-Right- | 560 |
| | gRNA12 | |
| 10 | Isogenic cells | 7224 |

Preparation of Embedded DNA Plugs from HEK293 Cells Culture

Agarose plugs with embedded DNA from isogenic or transfected HEK293 cells are prepared as described in Schurra and Bensimon (Schurra and Bensimon 2009). Briefly, cells were resuspended in 1×PBS at a concentration of 10⁷ cells/mL mixed thoroughly at a 1:1 ratio with a 1.2% w/v solution of low-melting point agarose (Nusieve GTG, ref. 50081, Cambrex) prepared in 1×PBS at 50° C. 90 μL of the cell/agarose mix was poured in a plug-forming well (BioRad, ref. 170-3713) and left to cool down at least 30 min at 4° C. Agarose plugs were incubated overnight at 50° C. in 250 μL of a 0.5M EDTA (pH 8), 1% Sarkosyl, 250 μg/mL proteinase K (Eurobio, code: GEXPRK01, France) solution, then washed twice in a Tris 10 mM, EDTA 1 mM solution for 30 in at room temperature.

Final Extraction of DNA and Molecular Combing

Plugs of embedded DNA from HEK293 control and transfected cells were treated for combing DNA as previously described (Schurra and Bensimon 2009). Briefly, plugs were melted at 68° C. in a MES 0.5 M (pH 5.5) solution for 20 min, and 1.5 units of beta-agarase (New England Biolabs, ref. M0392S, MA, USA) was added and left to incubate for up to 16 h at 42° C. The DNA solution was then poured in a Disposable DNA reservoir (Genomic Vision S.A., Paris, France) and Molecular Combing was performed using the Molecular Combing System (Genomic Vision S.A., Paris, France) and CombiCoverslips® (20 mm×20 mm, Genomic Vision S.A., Paris, France). The combed surfaces were dried for 4 hours at 60° C.

Synthesis and Labelling of BRCA Probes

The coordinates of the probes relative to the human GRCh37/hg19 sequence (chr17:41,176,611-41,372,447) are listed in table F. Probe size ranges from 3059 to 9551 bp in this example.

TABLE F
BRCA probes

| Probe ID | Chr | Start | End | Size |
|---|---|---|---|---|
| a1 | chr17 | 41176611 | 41185451 | 8840 |
| a2 | chr17 | 41185523 | 41194231 | 8708 |
| S1 | chr17 | 41195903 | 41203180 | 7277 |
| SEx21 | chr17 | 41205246 | 41211745 | 6499 |
| S2 | chr17 | 41215259 | 41223260 | 8001 |
| S3Big | chr17 | 41226181 | 41234768 | 8587 |
| S4 | chr17 | 41242909 | 41251961 | 9052 |
| S5 | chr17 | 41256140 | 41262844 | 6704 |
| S6 | chr17 | 41264546 | 41269110 | 4564 |
| Synt1 | chr17 | 41269785 | 41274269 | 4484 |
| S7 | chr17 | 41275398 | 41278706 | 3308 |
| S8 | chr17 | 41286084 | 41293383 | 7299 |
| S9 | chr17 | 41299811 | 41305857 | 6046 |
| b2 | chr17 | 41330367 | 41338479 | 8112 |
| b3 | chr17 | 41338628 | 41348179 | 9551 |
| S10 | chr17 | 41363153 | 41372447 | 9294 |
| Synt1b | chr17 | 41306593 | 41310952 | 4359 |
| S7b1 | chr17 | 41319666 | 41323534 | 3868 |
| S11_2 | chr17 | 41311309 | 41316264 | 4955 |
| S12_2 | chr17 | 41316540 | 41319599 | 3059 |

Except for the Synt1b, S7b_1, S11_2 and S12_2 probes, all probes were previously described in Cheeseman et al. (Cheeseman, Rouleau et al. 2012) and in WO2014/140788(A1). The Synt1b, S7b_1, S11_2 and S12_2 probes were produced by long-range PCR using LR Taq DNA polymerase (Roche, kit code: 11681842001) using the primers listed in table G and the Bacterial Artificial Chromosome (BAC) RP11-831F13 (Invitrogen) as template DNA. PCR products were ligated in the pCR-XL-TOPO® vector using the TOPO® XL PCR cloning Kit (Invitrogen, France, code K455010). The two extremities of each probe were sequenced for verification purpose.

TABLE G
PCR primer pairs used for BRCA probes cloning

| Probe | Primer | |
|---|---|---|
| Name | Name | Sequence (5′−>3′) |
| Synt1b | Synt1b_For | TTTAGAAAATACATCACCCCAGTTCC |
| | | (SEQ. ID NO: 20) |
| | Synt1b_Rev | TTGAAATACCACCTTTTCATTTCCAGA |
| | | (SEQ. ID NO: 21) |
| S7b_1 | S7b_For | GGAGGCAGAAATTGGGCATA |
| | | (SEQ. ID NO: 22) |
| | S7b_Rev | TTCTGACCCACAGACTCTCCA |
| | | (SEQ. ID NO: 23) |
| S11_2 | S11_For | CTCGATTCAAAAACAAAATGTGGCC |
| | | (SEQ. ID NO: 24) |
| | S11_Rev | ATGCCGTAGTTGGTCCAACG |
| | | (SEQ. ID NO: 25) |
| S12_2 | S12_For | AAAAACTCTACATCAGGGGACA |
| | | (SEQ. ID NO: 26) |
| | S12_Rev | AAAGAAAGAAAAAGTAAAAACTAAAGG |
| | | (SEQ. ID NO: 27) |

For labelling, the BRCA probes are grouped according to the incorporated hapten: probes a1+a2 (apparent B probe), SEx21 (apparent b probe), S3Big (apparent d probe), S8 (apparent I probe), S9 (apparent j probe) and b2 (apparent n probe) are jointly labelled with 3-Amino-3-Deoxydigoxigenin-9-dCTP (AminoDIG-9-dCTP); probes S1 (apparent a probe), S5 (apparent f probe), S7 (apparent h probe), S7b+12_2 (apparent 1 probe) and b3 (apparent m probe) are jointly labelled with Fluorescein-12-dUTP (Fluo-dUTP); probes S2 (apparent c probe), S4 (apparent e probe), S6+Synt1 (apparent g probe), Synt1b+S11_2 (apparent k probe) and S10 (apparent R probe) are jointly labelled with biotin-11-dCTP (Biot-dCTP). 200 ng of each BRCA probe group were labelled using conventional random priming protocols with the BioPrime® DNA kit (Invitrogen, code: 18094-011, CA, USA) according to the manufacturer's instructions except the dNTP mix from the kit was replaced by the mix specified in Table H and the labelling reaction was allowed to proceed overnight. After labelling, labelled product is purified with PureLink® PCR Purification Kit (ThermoFischer Scientific; Code K310001) according to the manufacturer's instructions.

TABLE H
dNTP mix used for BRCA probe labelling

| | Non-modified dNTPs | |
|---|---|---|
| | (Invitrogen, | |
| Labelling | ref. 10297-018) | Hapten-coupled dNTP |
| Fluo-dUTP | dATP, dCTP, | Fluorescein-12-dUTP 20 μM |
| | dGTP 40 μM | (Sigma Aldrich, code |
| | each dTTP 20 μM | 000000011373242910) |
| AminoDIG- | dATP, dTTP, | 3-Amino-3- |
| 9-dCTP | dGTP 40 μM | Deoxydigoxigenin-9-dCTP |
| | each dCTP 20 μM | 20 μM (Perkin Elmer, code |
| | | NEL562001EA) |
| Biot-dCTP | dATP, dTTP, | Biotin-11-dCTP |
| | dGTP 40 μM | 20 μM Perkin Elmer, |
| | each dCTP 20 μM | code NEL538001EA) |

Hybridization of BRCA1 GMC on Combed Genomic DNA and Detection

Subsequent steps were also performed essentially as previously described in Schurra and Bensimon, 2009 (Schurra and Bensimon 2009). Briefly, a mix of labelled probes (250 ng of each probe) were ethanol-precipitated together with 10 μg herring sperm DNA and 2.5 μg Human Cot-1 DNA (Invitrogen, ref. 15279-011, CA, USA), resuspended in 20 μL of hybridization buffer (50% formamide, 2×SSC, 0.5% SDS, 0.5% Sarkosyl, 10 mM NaCl, 30% Block-aid (Invitrogen, ref. B-10710, CA, USA). The probe solution and probes were heat-denatured together on the Hybridizer (Dako, ref. S2451) at 90° C. for 5 min and hybridization was left to proceed on the Hybridizer overnight at 37° C. Slides were washed 3 times in 60° C. pre-warmed 2×SSC solution for 5 min at room temperature. After the last washing steps, the hybridized coverslips were gradually dehydrated in 70%, 90% and 100% ethanol solution and air dried. For detection, 20 μL of the antibody solution diluted in Block-Aid® was added on the slide and covered with a combed coverslip and the slide was incubated in humid atmosphere at 37° C. for 20 min. Detection of the BRCA GMC was carried out using a Alexa Fluor® 647-coupled mouse monoclonal anti-digoxygenin (Jackson Immunoresearch, code 200-162-037) antibody in a 1:25 dilution for AminoDIG9-dCTP-labelled probes, a Cy3-coupled mouse monoclonal anti-Fluorescein (Jackson Immunoresearch, code 200-602-156) antibody in a 1:25 dilution for Fluo-dUTP-labelled probes and an BV480-coupled streptavidin (BD Biosciences, code 564876) in a 1:25 dilution for Biot-dCTP-labelled probes. The slides were then washed 3 times in a 2×SSC, 1% Tween20 solution for 3 min at room temperature and all glass coverslips were dehydrated in ethanol and air dried.

Analysis of BRCA Detected Signals

Hybridized-combed DNA from isogenic and transfected HEK293 cells preparation were scanned without any mounting medium using an inverted automated epifluorescence microscope, equipped with a 40× objective (FiberVision®, Genomic Vision S.A., Paris, France) and the signals were analyzed by an in house software (FiberStudio® BRCA, Genomic Vision S.A., Paris, France). For quantification of CRISPR-Cas9 gRNA-guided BRCA1 deletion, all fluorescent array signals composed of a least 3 probes and containing the apparent probe a and probe c are taking into account. The fluorescent signals where the apparent blue probe b is present between apparent probe a and c (normal allele; % ND) or absent (6.5 kb deletion; % D) are counted in both isogenic (iso) and transfected (trans) HEK293 cells. In these conditions, the global CRISPR/Cas9 RNA guided system efficiency is calculated as follows:

$$Efficacy ( \% ) = \frac{\% Dtrans - \% Diso}{\% NDiso} \times 100$$

All fluorescent arrays that do not correspond to either the normal BRCA1 GMC v5.2 or the edited BRCA1 (without the sequence of the apparent blue b probe) are considered as rearranged BRCA1 signals. The frequency of rearranged BRCA1 signal is calculated as follows:

$$Frequency ( \% ) = \frac{N rearranged BRCA 1}{N total BRCA 1} \times 100$$

Statistical analysis of data was performed a Two-sample test of proportions using normal approximation, using Benjamini-Hochberg adjustment for multiple testing.

Detection and Quantification of Gene Editing Events in BRCA1 Mediated by CRISPR-Cas9

The inventors have applied Molecular Combing on DNA extracted from HEK293 cells that has been transfected with gRNA pairs targeting the 3′ region of the BRCA1 gene (GRCh37/hg19 sequence: chr17: 41,176,611-41,372,447) as indicated in FIG. 2B and Table C and hybridized with the BRCA1 GMC (FIG. 2A).

To detect the presence of the 6-5 kb BRCA1 deletion induced by the CRISPR-Cas9 in the pool of transfected HEK cells, a PCR analysis with different primer pairs as described in Table D and showed in FIG. 2B using control and transfected HEH293 DNA as template. After amplification, reaction products are electrophoresed on a 2% agarose gel. Images of stained PCR products are then obtained and analyzed by visual comparison (FIG. 2C). An amplification product with the BRCA-Left-PCR-F and BRCA-Left-PCR-R primer pair used as positive control is observed in all DNA samples. For BRCA-Left-PCR-F and BRCA-Right-PCR-R primer pair that flanked the targeted BRCA site, the expected 7224 bp-amplification product is not amplified in the isogenic control since the PCR extension time is only 30 s whereas a shorter PCR products (between 490 and 651 bp depending on the gRNA combination, see table E) is obtained in samples with the expected editing events in the BRCA1 gene. These results indicate that the expected CRISPR-Cas9-mediated gene events are present in an undefined proportion of cells in the transfected HEK293 cells pool.

To visualize and quantify the BRCA1 6.5 kb-deletion induced by the CRIPSR-Cas9 system, the labelled BRCA1 specific probes were hybridized on combed DNA extracts from isogenic HEK293 cells (control) and in HEK293 cells transfected with the Left-gRNA7+BRCA-Right-gRNA4, Left-gRNA7+BRCA-Right-gRNA9 and Left-gRNA7+BRCA-Right-gRNA12 gRNA pairs. Immuno-fluorescence microscopy (FIG. 2D; aminoDIG9-labelled probes are represented by black boxes, Fluo- and Biot-labelled probes are depicted by grey and white boxes, respectively) exhibit between 238 and 740 multicolor linear patterns per conditions (Table I) that fulfilled the criteria for evaluation (see “Analysis of BRCA detected signals” section). No edited BRCA1 gene was detected in the isogenic HEK293 control cells whereas 10.5%, 11.1% and 6.5% of edited BRCA1 gene (where sequence b has been deleted) have been quantified in transfected HEK293 cells with the Left-gRNA7+BRCA-Right-gRNA4, Left-gRNA7+BRCA-Right-gRNA9 and Left-gRNA7+BRCA-Right-gRNA12 gRNA pairs, respectively (FIG. 2E). Statistical analysis showed that the observed proportion of gene editing events in transfected HEK293 cells is significant compared to the isogenic HEK293 control cells. It also showed that the Left-gRNA7+BRCA-Right-gRNA4 and Left-gRNA7+BRCA-Right-gRNA9 combinations exhibited a significant higher efficiency than Left-gRNA7+BRCA-Right-gRNA12 gRNA pairs.

The inventors have found that the Molecular Combing techniques of the invention are powerful methods for the detection of CRISPR-Cas9-induced gene editing events at the level of the unique molecule and to quantify its activity efficacy.

Detection and Quantification of Rearranged BRCA1 Gene Mediated by CRISPR-Cas9

The inventors detected fluorescent arrays (FIG. 2F; aminoDIG9-labelled probes are represented by black boxes, Fluo- and Biot-labelled probes are depicted by grey and white boxes, respectively) that do not correspond to the normal BRCA1 GMC v5.2 or to the edited BRCA1 form, e.g., with the deleted sequence corresponding to the apparent blue b probe, that probably arise from recombination induced by the CRISPR-Cas9 activity in transfected HEK293 cells with the gRNA pairs.

The labelled BRCA1 specific probes were hybridized on combed DNA extracts from isogenic HEK293 cells (control) and in HEK293 cells transfected with the Left-gRNA7+BRCA-Right-gRNA4, Left-gRNA7+BRCA-Right-gRNA9 and Left-gRNA7+BRCA-Right-gRNA12 gRNA pairs to evaluate the proportion of the non-canonical structures in the BRCA1 gene. A total of hybridization signals comprising between 238 and 740 fluorescent signals per condition were identified and classified. 0.9% of rearranged BRCA1 gene have been quantified in isogenic HK293 control cells whereas 3.8%, 2.5% and 1.6% of rearranged BRCA1 gene is detected in transfected HEK293 cells with the Left-gRNA7+BRCA-Right-gRNA4, Left-gRNA7+BRCA-Right-gRNA9 and Left-gRNA7+BRCA-Right-gRNA12 gRNA pairs, respectively (FIG. 2G and Table I). The increased frequency of rearranged BRCA1 gene in HEK293 cells transfected with the different gRNA pairs tested suggests that the designed CRISPR-Cas9 may induced other large rearrangements in BRCA1 than the expected ones, e.g., deletion of the sequence corresponding to the apparent blue b probe. Statistical analysis showed that the observed proportion of rearranged BRCA1 gene in transfected HEK293 cells with Left-gRNA7+BRCA-Right-gRNA9 and Left-gRNA7+BRCA-Right-gRNA12 gRNA pairs is not statistically different than the isogenic HEK293 control cells whereas this proportion is significantly higher for the Left-gRNA7+BRCA-Right-gRNA4 combination indicating that this last gRNA pairs is less specific than the two others (FIG. 2G).

Molecular Combing enables the visualization and the quantification of unexpected rearranged BRCA1 gene induced by CRISPR-Cas9 and by their infinity of combination of barcode possible is a powerful method to analyze and quantify them.

TABLE I
Summary of data.

| | Number of BRCA1 signals | | | | Frequencies (%) | | |
|---|---|---|---|---|---|---|---|
| Conditions | normal | edited | LR | total | normal | edited | LR |
| HEK293 isogenic | 442 | 0 | 4 | 446 | 99.1 | 0.0 | 0.9 |
| control | | | | | | | |
| BRCA-Left- | 204 | 25 | 9 | 238 | 85.7 | 10.5 | 3.8 |
| gRNA7 + BRCA- | | | | | | | |
| Right-gRNA4 | | | | | | | |
| BRCA-Left- | 381 | 49 | 11 | 441 | 86.4 | 11.1 | 2.5 |
| gRNA7 + BRCA- | | | | | | | |
| Right-gRNA9 | | | | | | | |
| BRCA-Left- | 680 | 48 | 12 | 740 | 91.9 | 6.5 | 1.6 |
| gRNA7 + BRCA- | | | | | | | |
| Right-gRNA12 | | | | | | | |

### Example 3—Detection and Quantification of Potential Off-Target Sites Induced by CRISPR-Cas9 RNA-Guided Nucleases

To identify potential off-target sites that might be generated by the different combinations of gRNA used to create a 6.5 kb deletion in the BRCA gene as described in Example 2, the inventors used the Cas-OFFinder (available online: http://_www.rgenome.net/cas-offinder/) that is an algorithm that quickly searches for possible off-target sites of Cas9 nucleases guided by gRNA. This CRIPSR recognition tool searches the entire genome for off-targeting and supports up to 10 mismatches and 7 different PAM types. In this example, the potential Off-target sites generated by the Cas9 from Streptococcus pyogenes with the 5′-NRG-3′ (R=A or G) sequence as PAM type in human GRCh37/hg19 sequence were identified with 2 mismatches at maximum. The results are shown in Table J.

TABLE J
Examples of potential Off-targets generated by the designed BRCA1 gRNA.
Abbreviations: Chr: Chromosome; Dir: Direction; Mis: Mismatches.

| gRNA | | crRNA | DNA target sequence | | | | | Bulge |
|---|---|---|---|---|---|---|---|---|
| combination | gRNA name | sequence (5′−>3′) | sequence (5′−>3′) | Chr. | Position | Dir. | Mis. | Size |
| 5 | BRCA-Left- | ATTCATACAGGTAGTGAGAGN | AaTCATACAGGTAGTGAcA | 3 | 166539742 | + | 2 | 0 |
| | gRNA7 | RG | GAAG | | | | | |
| | | (SEQ. ID NO: 28) | (SEQ. ID NO: 29) | | | | | |
| | | ATTCATACAGGTAGTGAGAGN | ATTCAgACAGGTAGaGAGA | 19 | 15530936 | + | 2 | 0 |
| | | RG | GGAG | | | | | |
| | | (SEQ. NO: 28) | (SEQ. ID NO: 30) | | | | | |
| | | ATTCATACAGGTAGTGAGAGN | ATTCATACAGGTAcTGtGA | 15 | 33022743 | + | 2 | 0 |
| | | RG | GAAG | | | | | |
| | | (SEQ. NO: 28) | (SEQ. ID NO: 31) | | | | | |
| | BRCA-Right- | CCACACCACCAATTACCACAN | CCACACCACCAATTACCAC | − | − | | − | − |
| | gRNA4 | RG | AAGG | | | | | |
| | | (SEQ. ID NO: 32) | (SEQ. ID NO: 33) | | | | | |
| 3 | BRCA-Left- | AATCATACAGGTAGTGAGAGN | AaTCATACAGGTAGTGAcA | 3 | 166539742 | + | 2 | 0 |
| | gRNA7 | RG | GAAG | | | | | |
| | | (SEQ. ID NO: 34) | (SEQ. ID NO: 35) | | | | | |
| | | AATCATACAGGTAGTGAGAGN | ATTCAgACAGGTAGaGAGA | 19 | 15530396 | + | 2 | 0 |
| | | RG | GGAG | | | | | |
| | | (SEQ. ID NO: 34) | (SEQ. ID NO: 36) | | | | | |
| | | AATCATACAGGTAGTGAGAGN | ATTCATACAGGTAcTGtGA | 15 | 33022743 | + | 2 | 0 |
| | | RG | GAAG | | | | | |
| | | (SEQ. ID NO: 34) | (SEQ. ID NO: 37) | | | | | |
| | BRCA-Right- | ATGGGAGAAGGTCATAGATGN | ATGGaAGAAGGTaATAGAT | 11 | 62891640 | + | 2 | 0 |
| | gRNA9 | RG | GAGG | | | | | |
| | | (SEQ. ID NO: 38) | (SEQ. ID NO: 39) | | | | | |
| 2 | BRCA-Left- | ATTCATACAGGTAGTGAGAGN | AaTCATACAGGTAGTGAcA | 3 | 166539742 | + | 2 | 0 |
| | gRNA7 | RG | GAAG | | | | | |
| | | (SEQ. ID NO: 40) | (SEQ. ID NO: 41) | | | | | |
| | | ATTCATACAGGTAGTGAGAGN | ATTCAgACAGGTAGaGAGA | 19 | 15530936 | + | 2 | 0 |
| | | RG | GGAG | | | | | |
| | | (SEQ. ID NO: 40) | (SEQ. ID NO: 42) | | | | | |
| | | ATTCATACAGGTAGTGAGAGN | ATTCATACAGGTAcTGtGA | 15 | 33022743 | + | 2 | 0 |
| | | RG | GAAG | | | | | |
| | | (SEQ. ID NO: 40) | (SEQ. ID NO: 43) | | | | | |
| | BRCA-Right- | GTGGAGGCAGAGATTACACAN | GTGGAGGCAGAGgcTACAC | 16 | 569309 | + | 2 | 0 |
| | gRNA12 | RG | ATGG | | | | | |
| | | (SEQ. ID NO: 44) | (SEQ. ID NO: 45) | | | | | |
| | | GTGGAGGCAGAGATTACACAN | GTGaAGGCAGAGgTTACAC | 1 | 883225944 | − | 2 | 0 |
| | | RG | AGGG | | | | | |
| | | (SEQ. ID NO: 44) | (SEQ. ID NO: 46) | | | | | |
| | | GTGGAGGCAGAGATTACACAN | GTtGAGGCAGtGATTACAC | 19 | 32828962 | + | 2 | 0 |
| | | RG | ATGG | | | | | |
| | | (SEQ. ID NO: 44) | (SEQ. ID NO: 47) | | | | | |
| | | GTGGAGGCAGAGATTACACAN | GaGtAGGCAGAGATTACAC | 10 | 36169278 | − | 2 | 0 |
| | | RG | AGGG | | | | | |
| | | (SEQ. ID NO: 44) | (SEQ. ID NO: 48) | | | | | |
| | | GTGGAGGCAGAGATTACACAN | ATGGAGtCAGAGATTACAC | 10 | 66905349 | − | 2 | 0 |
| | | RG | AAAG | | | | | |
| | | (SEQ. ID NO: 44) | (SEQ. ID NO: 49) | | | | | |
| | | GTGGAGGCAGAGATTACACAN | GTGGAGGCAGAGATTAgAg | 10 | 128209385 | − | 2 | 0 |
| | | RG | AGGG | | | | | |
| | | (SEQ. ID NO: 44) | (SEQ. ID NO: 50) | | | | | |

In a manner to analogous to the detection of large rearrangements in the BRCA1 gene induced by the CRISPR Cas9 system in Example 2 (FIGS. 2F and 2G), specific and unique GMCs are specially designed to cover each potential Off-target sites that have been identified. Molecular combing is performed using these specially designed probes to detect the different fluorescent arrays in cells treated with the CRISPR-Cas9 and isogenic cells used as control. The fluorescent arrays that do not correspond to the designed GMCs correspond to large rearrangements. By compared the control and treated cells, the frequency of these genomic events associated with the activity of the designed CRISPR-Cas9 system is determined.

ddPCR Characterization of the Transfected Cell Pools

The genomic DNA from isogenic or transfected HEK293 cells was subsequently used for a characterization of the targeted BRCA region with the QX200 Droplet Digital PCR (ddPCR™) System (Bio-Rad). The absolute quantification of the deletion events in the transfected versus the isogenic cells was performed with the ddPCR EvaGreen-based assay. The instrument control and the data analysis were carried out using the QuantaSoft™ Software (version 1.7). For each experimental point, 10 ng of genomic DNA were used in a final PCR reaction volume of 20 μl. The cycling conditions were 5 min at 95° C., and 35 cycles of 95° C. for 30 s, 65° C. for 1 min, followed by 5 min at 4° C. and a final denaturation step at 98° C. for 5 min (Eppendorf Nexus Gradient master cycler). The sequences and the Tm values of the two pairs of primers used in the PCR experiments (BRCA-Left-PCR-F/BRCA-Left-PCR-R and BRCA-Left-PCR-F/BRCA-Right-PCR-R; final concentration, 150 nM each) are described in Table D.

PCRs were analyzed with a QX200 droplet reader. The genomic DNAs prepared from HEK293 cells transfected with the BRCA-Left-gRNA7+BRCA-Right-gRNA4 and the BRCA-Left-gRNA7+BRCA-Right-gRNA9 gRNA pairs were analyzed in quadruplicates. DNAs extracted from the isogenic HEK293 cells (control) and from cells transfected with the BRCA-Left-gRNA7+BRCA-Right-gRNA12 gRNA pairs were analyzed in triplicates. For each sample, the number of copies of normal (N) and edited alleles (6.5 kb deletion; D) in both isogenic (iso) and transfected (trans) HEK293 cells are presented in Table K. Because of arbitrary threshold choices some PCR events are counted as deletions in isogenic controls. Thus, for each gRNA pair the CRISPR/Cas9 RNA guided system efficacy is calculated as follows:

$$Efficacy ( \% ) = [ mean ( \frac{D trans}{D trans + N trans} ) - mean ( \frac{D iso}{D iso + N iso} ) ] \times 100$$

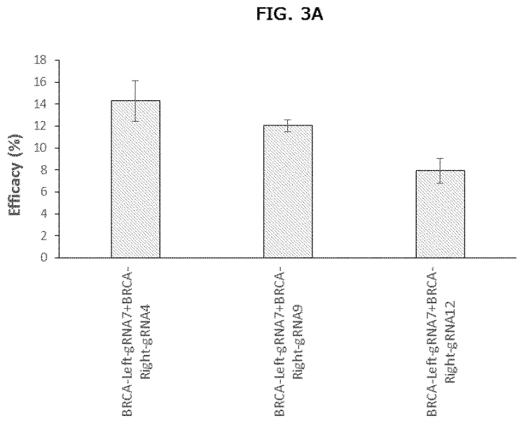

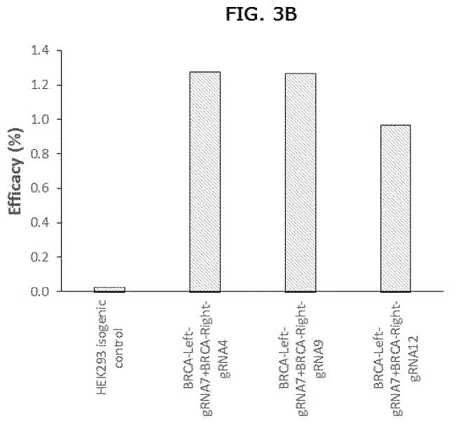

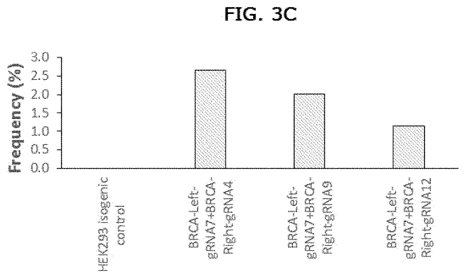

14.3±1.8%, 12.0±0.5% and 7.9±1.1% of edited BRCA1 gene (6.5 kb deletion) have been quantified in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs, respectively (FIG. 3A). These values are close to those calculated with the Molecular Combing technique but are systematically higher and present a lower standard deviation (FIG. 2E). The differences are probably due to the greater numbers of events analyzed by ddPCR (on average a total of 2059 events per sample was measured with ddPCR versus 466 with Molecular Combing). On the other hand, as PCR primers are located on both side of the expected deletion and close to the cutting sites, only bona fide deletion events are quantified by the ddPCR approach. To be detected and quantified, rearrangement events such as duplications and inversions, would necessitate the design of specific primers. In any case and in contrast to the Molecular Combing approach, the ddPCR technique would not be able to provide an exhaustive characterization and quantification of the unwanted events owing to an analysis centered on a narrow region around the cutting sites.

TABLE K
Summary of data.

| | Number of BRCA1 | | | Frequencies | |
|---|---|---|---|---|---|
| | events | | | (%) | |
| Conditions | Normal | Edited | Total | Normal | Edited |
| HEK293 isogenic | 1932 | 10.8 | 1942.8 | 99.4 | 0.6 |
| control | 1988 | 17.4 | 2005.4 | 99.1 | 0.9 |
| | 1942 | 28.4 | 1970.4 | 98.6 | 1.4 |
| BRCA-Left- | 1848 | 340 | 2188 | 84.5 | 15.5 |
| gRNA7 + BRCA- | 2202 | 332 | 2534 | 86.9 | 13.1 |
| Right-gRNA4 | 2190 | 466 | 2656 | 82.5 | 17.5 |
| | 2226 | 388 | 2614 | 85.2 | 14.8 |
| BRCA-Left- | 1450 | 224 | 1674 | 86.6 | 13.4 |
| gRNA7 + BRCA- | 1428 | 224 | 1652 | 86.4 | 13.6 |
| Right-gRNA9 | 1442 | 206 | 1648 | 87.5 | 12.5 |
| | 1392 | 200 | 1592 | 87.4 | 12.6 |
| BRCA-Left- | 1896 | 194 | 2090 | 90.7 | 9.3 |
| gRNA7 + BRCA- | 1774 | 190 | 1964 | 90.3 | 9.7 |
| Right-gRNA12 | 1878 | 154 | 2032 | 92.4 | 7.6 |

Characterization of the Transfected Pools of Cells by Targeted Next-Generation Sequencing (NGS)

Genomic DNAs from isogenic or transfected HEK293 cells were also used for targeted resequencing of the whole BRCA1 gene by NGS. One to 3 μg of each genomic DNA sample was mechanically fragmented with a Covaris focused-ultrasonicator (fragments median size: 200 bp). 100 ng of this fragmented DNA were end-labeled with 8 bases specific Illumina barcodes. Barcoded DNA fragments were then PCR amplified and a selective capture of the BRCA1 gene was performed on 750 ng of the PCR libraries using home-made biotinylated probes. The probes were designed to cover a 207 kb region on chromosome 17 containing the BRCA1 gene. The limits of the region are Chr17: 41,172,482-41,379,594 according to the GRCh37/hg19 assembly of the human reference genome. Single strand DNA molecules of the barcoded libraries, complementary to the biotinylated probes, were captured on streptavidin coated magnetic beads and subsequently amplified by PCR to generate a final pool of post capture libraries. Two independent post capture libraries were generated for each DNA sample extracted from isogenic or transfected HEK293 cells, respectively.

Post capture libraries were sequenced with the Illumina paired-end technology on a HiSeq2500 sequencing system. After demultiplexing, the FASTQ sequences files were aligned to the GRCh37/hg19 assembly of the human reference genome using the Burrows-Wheeler Aligner (Li, H. (2012) “Exploring single-sample SNP and INDEL calling with whole-genome de novo assembly.” Bioinformatics 28 (14): 1838-1844). The mean depth of coverage obtained for each sample was ≥2000×, with ≥100% of the targeted bases covered at least 100×.

For the quantification of deletions and unwanted events, only reads covering the chromosome 17: 41,205,189 location (corresponding to the breaking site targeted by the BRCA-Left-gRNA7 RNA guide and common to all three pairs of gRNA) and displaying a template >6000 bp were selected with the Sambamba tool. From these new BAM files a paired-end clustering analysis was carried out. For deletions, only the FR pairs (first read in forward orientation, second read in reverse orientation) were counted. FF and RR pairs, and RF pairs were considered, for the quantification of inversions and duplication events, respectively. For each sample, the number of copies of normal (N), deleted (Del), Inverted (Inv) and duplicated (Dup) alleles in both isogenic (iso) and transfected (trans) HEK293 cells are presented in Table L. The CRISPR/Cas9 RNA guided system efficiency is calculated as follows:

$$Efficacy ( \% ) = mean ( \frac{Del}{Total of events} ) \times 100$$

The frequency of rearranged BRCA1 alleles is calculated as follows:

$$Frequency ( \% ) = mean ( \frac{Inv + Dup}{Total of events} ) \times 100$$

The deletions frequencies, as measured by NGS, are 1.3%, 1.3% and 1% in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs, respectively (FIG. 3. B). These values are about ten times lower than those calculated with the Molecular Combing and the ddPCR approaches (FIG. 3B and FIG. 2E). This discrepancy might be due to an experimental bias during the targeted capture of the BRCA1 gene with oligonucleotides biotinylated probes and streptavidin-coated magnetic beads. Actually, the efficiency of the specific capture of the BRCA1 sequences is not known. Furthermore, the two mandatory PCR steps of the targeted NGS protocol are probably a source of errors too.

In contrast to results obtained for deletions, the frequencies of rearrangements in HEK293 cells transfected with the BRCA-Left-gRNA7+BRCA-Right-gRNA4, the BRCA-Left-gRNA7+BRCA-Right-gRNA9 and the BRCA-Left-gRNA7+BRCA-Right-gRNA12 gRNA pairs are in the same order of magnitude as those calculated with the Molecular Combing technique: 2.6%, 2% and 1.1% versus 3.8%, 2.5% and 1.6%, respectively (FIG. 3C and FIG. 2G).

Compared to the two tested alternative approaches (absolute quantification by ddPCR and targeted next-generation sequencing) the Molecular Combing technique is unique in that it enables a reliable and rapid detection and quantification of deletions induced by engineered nucleases in the BRCA1 gene, as well as unwanted large rearrangements. This advantage is notably due to the possibility to visualize and analyze a large genomic region around the sites targeted by programmable nucleases. On the other hand, the major advantage of the Molecular Combing technique is the absence of amplification steps in the course of the protocol, amplifications which are potential sources of statistical errors. This unbiased method, by analyzing long and unique DNA molecules, allows the selection and the validation of the engineered cells presenting the expected editing events and the rejection of cells harboring unwanted rearrangements.

TABLE L
Summary of data.

| | Number of BRCA1 events | | | | |
|---|---|---|---|---|---|
| | | Deletion | Inversion | Duplication | |
| conditions | Normal | (FR) | (FF and RR) | (RF) | Total |
| HEK293 | 2085 | 1 | 0 | 0 | 2086 |
| isogenic control | 1988 | 0 | 0 | 0 | 1988 |
| BRCA-Left- | 1332 | 18 | 39 | 5 | 1394 |
| gRNA7 + BRCA- | 1537 | 20 | 30 | 4 | 1591 |
| Right-gRNA4 | | | | | |
| BRCA-Left- | 1695 | 20 | 29 | 7 | 1751 |
| gRNA7 + BRCA- | 1814 | 26 | 28 | 8 | 1876 |
| Right-gRNA9 | | | | | |
| BRCA-Left- | 1615 | 17 | 19 | 1 | 1652 |
| gRNA7 + BRCA- | 1621 | 15 | 13 | 4 | 1653 |
| Right-gRNA12 | | | | | |

Stringent Conditions of Hybridization of Probes Covering the BRCA1 Gene in the Molecular Combing Approach.

The procedures for the synthesis and the labelling of the probes covering the BRCA1 locus are precisely described in the “Synthesis and labelling of BRCA1 probes” section of the Example 2 paragraph.

The next section—“Hybridization of BRCA1 GMC on combed genomic DNA and detection”—deals with the hybridization of the probes and the detection of the region of interest. As mentioned, the high stringency of the hybridizations conditions is provided by both the salinity of the hybridization buffer, the presence of ionic surfactants and the use of formamide (50% formamide, 2×SSC, 0.5% SDS, 0.5% Sarkosyl, 10 mM NaCl, 30% Block-aid (Invitrogen, ref. B-10710, CA, USA). In addition, the specificity of the DNA probes is strengthened by the use of herring sperm DNA which reduces non-specific binding to the surface of the cover-slip. Furthermore, the Human Cot-1 DNA limits the unspecific hybridization of the probes synthesized by random-priming to the repetitive elements scattered through the genome. Finally, after the hybridization step, the coverslips are washed three times at 60° C. for 5 min in 2×SSC to eliminate non-specific binding. All that experimental conditions contribute to the high stringency of the hybridizations carried out on combed DNA fibers.

Detecting and Quantifying Unexpected or Unwanted Rearrangements or Genetic Events.

The labelled Genomic Morse Code sequences, as defined as a general technology in the present invention, are designed to cover the genomic region and/or the gene to be edited by the engineered nucleases or the mega-nucleases. In the case of the BRCA1 gene engineering, the total length of the probes constituting the GMC is equal to 132,567 bases (see FIGS. 2A. and 2B. and Table F.) and far exceeds the 82.1 kb of the gene. Preferentially, one of the probes constituting the GMC covers the region to be edited. This is notably the case in the BRCA1 experiments where the b probe approximately corresponds to the 6.5 kb deletion induced by the CRISPR-cas9 system (see FIGS. 2A. and 2B.). The detection of the deletion (6.5 kb) and the measure of the nucleases efficiency are carried out by comparing the profile of the GMC in the engineered cells to the reference profile in the isogenic (control) non-transfected cells. In a word, the b probe of the BRCA1 GMC is detectable in the control cells and absent in the cells correctly edited by the engineered nucleases. By extension, any GMC profile not corresponding to those expected either in the isogenic (control) or the edited (deletion) cells is the signature of an unwanted event. Such a rearrangement is presented in FIG. 2F. This inversion/duplication event can be due to only one cut instead of two (the two sgRNA pairs did not work simultaneously) and to an homologous recombination at the probe b level.

### Terminology

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by deletion of http: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all subranges or intermediate values subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

### SCIENTIFIC PUBLICATIONS

Aach, J., P. Mali, et al. (2014). “CasFinder: Flexible algorithm for identifying specific Cas9 targets in genomes.” bioRxiv.
Abudayyeh, O. O., J. S. Gootenberg, et al. (2016). “C2c2 is a single-component programmable RNA-guided RNA-targeting CRISPR effector.” Science 353(6299): aaf5573.
Allers, T. and M. Lichten (2001). “Differential timing and control of noncrossover and crossover recombination during meiosis.” Cell 106(1): 47-57.
Allers, T. and M. Lichten (2001). “Intermediates of yeast meiotic recombination contain heteroduplex DNA.” Mol Cell 8(1): 225-231.
Arnould, S., C. Perez, et al. (2007). “Engineered I-CreI derivatives cleaving sequences from the human XPC gene can induce highly efficient gene correction in mammalian cells.” J Mol Biol 371(1): 49-65.
Aronin, N. and M. DiFiglia (2014). “Huntingtin-lowering strategies in Huntington's disease: antisense oligonucleotides, small RNAs, and gene editing.” Mov Disord 29(11): 1455-1461.
Bae, S., J. Park, et al. (2014). “Cas-OFFinder: a fast and versatile algorithm that searches for potential off-target sites of Cas9 RNA-guided endonucleases.” Bioinformatics 30(10): 1473-1475.
Bailis, J. M., D. D. Luche, et al. (2008). “Minichromosome maintenance proteins interact with checkpoint and recombination proteins to promote s-phase genome stability.” Mol Cell Biol 28(5): 1724-1738.
Baker, M. (2012). “Gene-editing nucleases.” Nat Methods 9(1): 23-26.
Barrangou, R., C. Fremaux, et al. (2007). “CRISPR provides acquired resistance against viruses in prokaryotes.” Science 315(5819): 1709-1712.
Baum, C., U. Modlich, et al. (2011). “Concise review: managing genotoxicity in the therapeutic modification of stem cells.” Stem Cells 29(10): 1479-1484.
Beisel, C. L., A. A. Gomaa, et al. (2014). “A CRISPR design for next-generation antimicrobials.” Genome Biol 15(11): 516.
Belfort, M. and R. J. Roberts (1997). “Homing endonucleases: keeping the house in order.” Nucleic Acids Res 25(17): 3379-3388.
Bell, C. C., G. W. Magor, et al. (2014). “A high-throughput screening strategy for detecting CRISPR-Cas9 induced mutations using next-generation sequencing.” BMC Genomics 15: 1002.
Bhattacharyya, A. and D. M. Lilley (1989). “Single base mismatches in DNA. Long- and short-range structure probed by analysis of axis trajectory and local chemical reactivity.” J Mol Biol 209(4): 583-597.
Bibikova, M., D. Carroll, et al. (2001). “Stimulation of homologous recombination through targeted cleavage by chimeric nucleases.” Mol Cell Biol 21(1): 289-297.
Bibikova, M., M. Golic, et al. (2002). “Targeted chromosomal cleavage and mutagenesis in Drosophila using zinc-finger nucleases.” Genetics 161(3): 1169-1175.
Boch, J., H. Scholze, et al. (2009). “Breaking the code of DNA binding specificity of TAL-type III effectors.” Science 326(5959): 1509-1512.
Caburet, S., C. Conti, et al. (2005). “Human ribosomal RNA gene arrays display a broad range of palindromic structures.” Genome Res 15(8): 1079-1085.
Canver, M. C., D. E. Bauer, et al. (2014). “Characterization of genomic deletion efficiency mediated by clustered regularly interspaced palindromic repeats (CRISPR)/Cas9 nuclease system in mammalian cells.” J Biol Chem 289(31): 21312-21324.
Chapman, J. R., M. R. Taylor, et al. (2012). “Playing the end game: DNA double-strand break repair pathway choice.” Mol Cell 47(4): 497-510.
Cheeseman, K., J. Ropars, et al. (2014). “Multiple recent horizontal transfers of a large genomic region in cheese making fungi.” Nat Commun 5: 2876.
Cheeseman, K., E. Rouleau, et al. (2012). “A diagnostic genetic test for the physical mapping of germline rearrangements in the susceptibility breast cancer genes BRCA1 and BRCA2.” Hum Mutat 33(6): 998-1009.
Chen, B. and B. Huang (2014). “Imaging genomic elements in living cells using CRISPR/Cas9.” Methods Enzymol 546: 337-354.
Chevalier, B. S. and B. L. Stoddard (2001). “Homing endonucleases: structural and functional insight into the catalysts of intron/intein mobility.” Nucleic Acids Res 29(18): 3757-3774.
Cho, S. W., S. Kim, et al. (2013). “Targeted genome engineering in human cells with the Cas9 RNA-guided endonuclease.” Nat Biotechnol 31(3): 230-232.
Choulika, A., A. Perrin, et al. (1995). “Induction of homologous recombination in mammalian chromosomes by using the I-SceI system of Saccharomyces cerevisiae.” Mol Cell Biol 15(4): 1968-1973.
Christian, M., T. Cermak, et al. (2010). “Targeting DNA double-strand breaks with TAL effector nucleases.” Genetics 186(2): 757-761.
Chylinski, K., K. S. Makarova, et al. (2014). “Classification and evolution of type II CRISPR-Cas systems.” Nucleic Acids Res 42(10): 6091-6105.
Cicalese, M. P. and A. Aiuti (2015). “Clinical applications of gene therapy for primary immunodeficiencies.” Hum Gene Ther 26(4): 210-219.
Cohen-Tannoudji, M., S. Robine, et al. (1998). “I-SceI-induced gene replacement at a natural locus in embryonic stem cells.” Mol Cell Biol 18(3): 1444-1448.
Cong, L., F. A. Ran, et al. (2013). “Multiplex genome engineering using CRISPR/Cas systems.” Science 339(6121): 819-823.
Conti, C., J. Herrick, et al. (2007). “Unscheduled DNA replication origin activation at inserted HPV 18 sequences in a HPV-18/MYC amplicon.” Genes Chromosomes Cancer 46(8): 724-734.
Cradick, T. J., E. J. Fine, et al. (2013). “CRISPR/Cas9 systems targeting beta-globin and CCR5 genes have substantial off-target activity.” Nucleic Acids Res 41(20): 9584-9592.
D'Agostino, Y., A. Locascio, et al. (2016). “A Rapid and Cheap Methodology for CRISPR/Cas9 Zebrafish Mutant Screening.” Mol Biotechnol 58(1): 73-78.
Daboussi, F., S. Courbet, et al. (2008). “A homologous recombination defect affects replication-fork progression in mammalian cells.” J Cell Sci 121(Pt 2): 162-166.
Deltcheva, E., K. Chylinski, et al. (2011). “CRISPR RNA maturation by trans-encoded small RNA and host factor RNase III.” Nature 471(7340): 602-607.
Dong, D., K. Ren, et al. (2016). “The crystal structure of Cpf1 in complex with CRISPR RNA.” Nature 532(7600): 522-526.
Dorn, E. S., P. D. Chastain, 2nd, et al. (2009). “Analysis of re-replication from deregulated origin licensing by DNA fiber spreading.” Nucleic Acids Res 37(1): 60-69.
Doyon, Y., J. M. McCammon, et al. (2008). “Heritable targeted gene disruption in zebrafish using designed zinc-finger nucleases.” Nat Biotechnol 26(6): 702-708.
Dupuy, A., J. Valton, et al. (2013). “Targeted gene therapy of xeroderma pigmentosum cells using meganuclease and TALEN.” PLoS One 8(11): e78678.
Fonfara, I., H. Richter, et al. (2016). “The CRISPR-associated DNA-cleaving enzyme Cpf1 also processes precursor CRISPR RNA.” Nature 532(7600): 517-521.
Fu, Y., J. A. Foden, et al. (2013). “High-frequency off-target mutagenesis induced by CRISPR-Cas nucleases in human cells.” Nat Biotechnol 31(9): 822-826.
Gabriel, R., A. Lombardo, et al. (2011). “An unbiased genome-wide analysis of zinc-finger nuclease specificity.” Nat Biotechnol 29(9): 816-823.
Gad, S., A. Aurias, et al. (2001). “Color bar coding the BRCA1 gene on combed DNA: a useful strategy for detecting large gene rearrangements.” Genes Chromosomes Cancer 31(1): 75-84.
Gad, S., I. Bieche, et al. (2003). “Characterisation of a 161 kb deletion extending from the NBR1 to the BRCA1 genes in a French breast-ovarian cancer family.” Hum Mutat 21(6): 654.
Gad, S., V. Caux-Moncoutier, et al. (2002). “Significant contribution of large BRCA1 gene rearrangements in 120 French breast and ovarian cancer families.” Oncogene 21(44): 6841-6847.
Gad, S., M. Klinger, et al. (2002). “Bar code screening on combed DNA for large rearrangements of the BRCA1 and BRCA2 genes in French breast cancer families.” J Med Genet 39(11): 817-821.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-2586.
Grizot, S., J. Smith, et al. (2009). “Efficient targeting of a SCID gene by an engineered single-chain homing endonuclease.” Nucleic Acids Res 37(16): 5405-5419.
Grosse, S., N. Huot, et al. (2011). “Meganuclease-mediated Inhibition of HSV1 Infection in Cultured Cells.” Mol Ther 19(4): 694-702.
Gu, W. G. (2015). “Genome editing-based HIV therapies.” Trends Biotechnol 33(3): 172-179.
Guell, M., L. Yang, et al. (2014). “Genome editing assessment using CRISPR Genome Analyzer (CRISPR-GA).” Bioinformatics 30(20): 2968-2970.
Gueroui, Z., C. Place, et al. (2002). “Observation by fluorescence microscopy of transcription on single combed DNA.” Proc Natl Acad Sci USA 99(9): 6005-6010.
Guschin, D. Y., A. J. Waite, et al. (2010). “A rapid and general assay for monitoring endogenous gene modification.” Methods Mol Biol 649: 247-256.
Hale, C. R., S. Majumdar, et al. (2012). “Essential features and rational design of CRISPR RNAs that function with the Cas RAMP module complex to cleave RNAs.” Mol Cell 45(3): 292-302.
Hale, C. R., P. Zhao, et al. (2009). “RNA-guided RNA cleavage by a CRISPR RNA-Cas protein complex.” Cell 139(5): 945-956.
Hastings, P. J., J. R. Lupski, et al. (2009). “Mechanisms of change in gene copy number.” Nat Rev Genet 10(8): 551-564.
Heigwer, F., G. Kerr, et al. (2014). “E-CRISP: fast CRISPR target site identification.” Nat Methods 11(2): 122-123.
Hendel, A., E. J. Kildebeck, et al. (2014). “Quantifying genome-editing outcomes at endogenous loci with SMRT sequencing.” Cell Rep 7(1): 293-305.
Herrick, J. and A. Bensimon (1999). “Single molecule analysis of DNA replication.” Biochimie 81(8-9): 859-871.
Herrick, J., C. Conti, et al. (2005). “Genomic organization of amplified MYC genes suggests distinct mechanisms of amplification in tumorigenesis.” Cancer Res 65(4): 1174-1179.
Herrick, J., S. Jun, et al. (2002). “Kinetic model of DNA replication in eukaryotic organisms.” J Mol Biol 320(4): 741-750.
Herrick, J., X. Michalet, et al. (2000). “Quantifying single gene copy number by measuring fluorescent probe lengths on combed genomic DNA.” Proc Natl Acad Sci USA 97(1): 222-227.
Herrick, J., P. Stanislawski, et al. (2000). “Replication fork density increases during DNA synthesis in X. laevis egg extracts.” J Mol Biol 300(5): 1133-1142.
Hindson, C. M., J. R. Chevillet, et al. (2013). “Absolute quantification by droplet digital PCR versus analog real-time PCR.” Nat Methods 10(10): 1003-1005.
Hirano, H., J. S. Gootenberg, et al. (2016). “Structure and Engineering of Francisella novicida Cas9.” Cell 164(5): 950-961.
Hsu, P. D., D. A. Scott, et al. (2013). “DNA targeting specificity of RNA-guided Cas9 nucleases.” Nat Biotechnol 31(9): 827-832.
Huang, P., A. Xiao, et al. (2011). “Heritable gene targeting in zebrafish using customized TALENs.” Nat Biotechnol 29(8): 699-700.
Iyer, V., B. Shen, et al. (2015). “Off-target mutations are rare in Cas9-modified mice.” Nat Methods 12(6): 479.
Jinek, M., K. Chylinski, et al. (2012). “A programmable dual-RNA-guided DNA endonuclease in adaptive bacterial immunity.” Science 337(6096): 816-821.
Jo, Y. I., H. Kim, et al. (2015). “Recent developments and clinical studies utilizing engineered zinc finger nuclease technology.” Cell Mol Life Sci 72(20): 3819-3830.
Jun, S., J. Herrick, et al. (2004). “Persistence length of chromatin determines origin spacing in Xenopus early-embryo DNA replication: quantitative comparisons between theory and experiment.” Cell Cycle 3(2): 223-229.
Kim, Y. G., J. Cha, et al. (1996). “Hybrid restriction enzymes: zinc finger fusions to Fok I cleavage domain.” Proc Natl Acad Sci USA 93(3): 1156-1160.
Kim, Y. G. and S. Chandrasegaran (1994). “Chimeric restriction endonuclease.” Proc Natl Acad Sci USA 91(3): 883-887.
Kuscu, C., S. Arslan, et al. (2014). “Genome-wide analysis reveals characteristics of off-target sites bound by the Cas9 endonuclease.” Nat Biotechnol 32(7): 677-683.
Lebofsky, R. and A. Bensimon (2003). “Single DNA molecule analysis: applications of molecular combing.” Brief Funct Genomic Proteomic 1(4): 385-396.
Lebofsky, R. and A. Bensimon (2005). “DNA replication origin plasticity and perturbed fork progression in human inverted repeats.” Mol Cell Biol 25(15): 6789-6797.
Lebofsky, R., R. Heilig, et al. (2006). “DNA replication origin interference increases the spacing between initiation events in human cells.” Mol Biol Cell 17(12): 5337-5345.
Lee, H. J., J. Kweon, et al. (2012). “Targeted chromosomal duplications and inversions in the human genome using zinc finger nucleases.” Genome Res 22(3): 539-548.
Li, H. (2012) “Exploring single-sample SNP and INDEL calling with whole-genome de novo assembly.” Bioinformatics 28(14): 1838-1844
Li, L., L. P. Wu, et al. (1992). “Functional domains in Fok I restriction endonuclease.” Proc Natl Acad Sci USA 89(10): 4275-4279.
Lieber, M. R. (2010). “The mechanism of double-strand DNA break repair by the nonhomologous DNA end-joining pathway.” Annu Rev Biochem 79: 181-211.
Lieber, M. R. and T. E. Wilson (2010). “SnapShot: Nonhomologous DNA end joining (NHEJ).” Cell 142(3): 496-496 e491.
Llorente, B., C. E. Smith, et al. (2008). “Break-induced replication: what is it and what is it for?” Cell Cycle 7(7): 859-864.
Maeder, M. L. and C. A. Gersbach (2016). “Genome-editing Technologies for Gene and Cell Therapy.” Mol Ther 24(3): 430-446.
Maeder, M. L., S. J. Linder, et al. (2013). “CRISPR RNA-guided activation of endogenous human genes.” Nat Methods 10(10): 977-979.
Mahiet, C., A. Ergani, et al. (2012). “Structural variability of the herpes simplex virus 1 genome in vitro and in vivo.” J Virol 86(16): 8592-8601.
Makarova, K. S., Y. I. Wolf, et al. (2015). “An updated evolutionary classification of CRISPR-Cas systems.” Nat Rev Microbiol 13(11): 722-736.
Mali, P., J. Aach, et al. (2013). “CAS9 transcriptional activators for target specificity screening and paired nickases for cooperative genome engineering.” Nat Biotechnol 31(9): 833-838.
Mali, P., L. Yang, et al. (2013). “RNA-guided human genome engineering via Cas9.” Science 339(6121): 823-826.
Maresca, M., V. G. Lin, et al. (2013). “Obligate ligation-gated recombination (ObLiGaRe): custom-designed nuclease-mediated targeted integration through nonhomologous end joining.” Genome Res 23(3): 539-546.
Marraffini, L. A. and E. J. Sontheimer (2010). “Self versus non-self discrimination during CRISPR RNA-directed immunity.” Nature 463(7280): 568-571.
Mashal, R. D., J. Koontz, et al. (1995). “Detection of mutations by cleavage of DNA heteroduplexes with bacteriophage resolvases.” Nat Genet 9(2): 177-183.
McEachern, M. J. and J. E. Haber (2006). “Break-induced replication and recombinational telomere elongation in yeast.” Annu Rev Biochem 75: 111-135.
McMahon, M. A., M. Randar, et al. (2012). “Gene editing: not just for translation anymore.” Nat Methods 9(1): 28-31.
Merkert, S. and U. Martin (2016). “Targeted genome engineering using designer nucleases: State of the art and practical guidance for application in human pluripotent stem cells.” Stem Cell Res 16(2): 377-386.
Michalet, X., R. Ekong, et al. (1997). “Dynamic molecular combing: stretching the whole human genome for high-resolution studies.” Science 277(5331): 1518-1523.
Miller, J. C., S. Tan, et al. (2011). “A TALE nuclease architecture for efficient genome editing.” Nat Biotechnol 29(2): 143-148.
Mock, U., I. Hauber, et al. (2016). “Digital PCR to assess gene-editing frequencies (GEF-dPCR) mediated by designer nucleases.” Nat Protoc 11(3): 598-615.
Moehle, E. A., J. M. Rock, et al. (2007). “Targeted gene addition into a specified location in the human genome using designed zinc finger nucleases.” Proc Natl Acad Sci USA 104(9): 3055-3060.
Moscou, M. J. and A. J. Bogdanove (2009). “A simple cipher governs DNA recognition by TAL effectors.” Science 326(5959): 1501.
Mussolino, C., J. Alzubi, et al. (2014). “TALENs facilitate targeted genome editing in human cells with high specificity and low cytotoxicity.” Nucleic Acids Res 42(10): 6762-6773.
Nguyen, K., P. Walrafen, et al. (2011). “Molecular combing reveals allelic combinations in facioscapulohumeral dystrophy.” Ann Neurol 70(4): 627-633.
Oliveros, J. C., M. Franch, et al. (2016). “Breaking-Cas-interactive design of guide RNAs for CRISPR-Cas experiments for ENSEMBL genomes.” Nucleic Acids Res 44(W1): W267-271.
Osborn, M. J., B. R. Webber, et al. (2016). “Evaluation of TCR Gene Editing Achieved by TALENs, CRISPR/Cas9, and megaTAL Nucleases.” Mol Ther 24(3): 570-581.
Ousterout, D. G., P. Perez-Pinera, et al. (2013). “Reading frame correction by targeted genome editing restores dystrophin expression in cells from Duchenne muscular dystrophy patients.” Mol Ther 21(9): 1718-1726.
Paques, F. and J. E. Haber (1999). “Multiple pathways of recombination induced by double-strand breaks in Saccharomyces cerevisiae.” Microbiol Mol Biol Rev 63(2): 349-404.
Pasero, P., A. Bensimon, et al. (2002). “Single-molecule analysis reveals clustering and epigenetic regulation of replication origins at the yeast rDNA locus.” Genes Dev 16(19): 2479-2484.
Patel, P. K., B. Arcangioli, et al. (2006). “DNA replication origins fire stochastically in fission yeast.” Mol Biol Cell 17(1): 308-316.
Pattanayak, V., S. Lin, et al. (2013). “High-throughput profiling of off-target DNA cleavage reveals RNA-programmed Cas9 nuclease specificity.” Nat Biotechnol 31(9): 839-843.
Pattanayak, V., C. L. Ramirez, et al. (2011). “Revealing off-target cleavage specificities of zinc-finger nucleases by in vitro selection.” Nat Methods 8(9): 765-770.
Payen, C., R. Koszul, et al. (2008). “Segmental duplications arise from Pol32-dependent repair of broken forks through two alternative replication-based mechanisms.” PLoS Genet 4(9): e1000175.
Perez, E. E., J. Wang, et al. (2008). “Establishment of HIV-1 resistance in CD4+ T cells by genome editing using zinc-finger nucleases.” Nat Biotechnol 26(7): 808-816.
Pinheiro, L. B., V. A. Coleman, et al. (2012). “Evaluation of a droplet digital polymerase chain reaction format for DNA copy number quantification.” Anal Chem 84(2): 1003-1011.
Porter, S. N., L. C. Baker, et al. (2014). “Lentiviral and targeted cellular barcoding reveals ongoing clonal dynamics of cell lines in vitro and in vivo.” Genome Biol 15(5): R75.
Porteus, M. H. and D. Baltimore (2003). “Chimeric nucleases stimulate gene targeting in human cells.” Science 300(5620): 763.
Pruett-Miller, S. M., J. P. Connelly, et al. (2008). “Comparison of zinc finger nucleases for use in gene targeting in mammalian cells.” Mol Ther 16(4): 707-717.
Puchta, H. (2005). “The repair of double-strand breaks in plants: mechanisms and consequences for genome evolution.” J Exp Bot 56(409): 1-14.
Qiu, P., H. Shandilya, et al. (2004). “Mutation detection using Surveyor nuclease.” Biotechniques 36(4): 702-707.
Ran, F. A., L. Cong, et al. (2015). “In vivo genome editing using Staphylococcus aureus Cas9.” Nature 520(7546): 186-191.
Ran, F. A., P. D. Hsu, et al. (2013). “Double nicking by RNA-guided CRISPR Cas9 for enhanced genome editing specificity.” Cell 154(6): 1380-1389.
Rao, V. A., C. Conti, et al. (2007). “Endogenous gamma-H2AX-ATM-Chk2 checkpoint activation in Bloom's syndrome helicase deficient cells is related to DNA replication arrested forks.” Mol Cancer Res 5(7): 713-724.
Redondo, P., J. Prieto, et al. (2008). “Molecular basis of xeroderma pigmentosum group C DNA recognition by engineered meganucleases.” Nature 456(7218): 107-111.
Rouet, P., F. Smih, et al. (1994). “Expression of a site-specific endonuclease stimulates homologous recombination in mammalian cells.” Proc Natl Acad Sci USA 91(13): 6064-6068.
Sampson, T. R., S. D. Saroj, et al. (2013). “A CRISPR/Cas system mediates bacterial innate immune evasion and virulence.” Nature 497(7448): 254-257.
Schmid-Burgk, J. L., T. Schmidt, et al. (2014). “OutKnocker: a web tool for rapid and simple genotyping of designer nuclease edited cell lines.” Genome Res 24(10): 1719-1723.
Schurra, C. and A. Bensimon (2009). “Combing genomic DNA for structural and functional studies.” Methods Mol Biol 464: 71-90.
Shendure, J. and H. Ji (2008). “Next-generation DNA sequencing.” Nat Biotechnol 26(10): 1135-1145.
Shinkuma, S., Z. Guo, et al. (2016). “Site-specific genome editing for correction of induced pluripotent stem cells derived from dominant dystrophic epidermolysis bullosa.” Proc Natl Acad Sci USA.
Smih, F., P. Rouet, et al. (1995). “Double-strand breaks at the target locus stimulate gene targeting in embryonic stem cells.” Nucleic Acids Res 23(24): 5012-5019.
Smith, C., A. Gore, et al. (2014). “Whole-genome sequencing analysis reveals high specificity of CRISPR/Cas9 and TALEN-based genome editing in human iPSCs.” Cell Stem Cell 15(1): 12-13.
Sollu, C., K. Pars, et al. (2010). “Autonomous zinc-finger nuclease pairs for targeted chromosomal deletion.” Nucleic Acids Res 38(22): 8269-8276.
Sugawara, N., G. Ira, et al. (2000). “DNA length dependence of the single-strand annealing pathway and the role of Saccharomyces cerevisiae RAD59 in double-strand break repair.” Mol Cell Biol 20(14): 5300-5309.
Szostak, J. W., T. L. Orr-Weaver, et al. (1983). “The double-strand-break repair model for recombination.” Cell 33(1): 25-35.
Takata, M., M. S. Sasaki, et al. (1998). “Homologous recombination and non-homologous end-joining pathways of DNA double-strand break repair have overlapping roles in the maintenance of chromosomal integrity in vertebrate cells.” EMBO J 17(18): 5497-5508.
Taylor, G. R. and J. Deeble (1999). “Enzymatic methods for mutation scanning.” Genet Anal 14(5-6): 181-186.
Tessereau, C., M. Buisson, et al. (2013). “Direct visualization of the highly polymorphic RNU2 locus in proximity to the BRCA1 gene.” PLoS One 8(10): e76054.
Tessereau, C., M. Leone, et al. (2015). “Occurrence of a non deleterious gene conversion event in the BRCA1 gene.” Genes Chromosomes Cancer 54(10): 646-652.
Tessereau, C., Y. Lesecque, et al. (2014). “Estimation of the RNU2 macrosatellite mutation rate by BRCA1 mutation tracing.” Nucleic Acids Res 42(14): 9121-9130.
Thomas, H. R., S. M. Percival, et al. (2014). “High-throughput genome editing and phenotyping facilitated by high resolution melting curve analysis.” PLoS One 9(12): e114632.
Tones, R., M. C. Martin, et al. (2014). “Engineering human tumour-associated chromosomal translocations with the RNA-guided CRISPR-Cas9 system.” Nat Commun 5: 3964.
Triques, K., E. Piednoir, et al. (2008). “Mutation detection using ENDO1: application to disease diagnostics in humans and TILLING and Eco-TILLING in plants.” BMC Mol Biol 9: 42.
Tsai, S. Q., Z. Zheng, et al. (2015). “GUIDE-seq enables genome-wide profiling of off-target cleavage by CRISPR-Cas nucleases.” Nat Biotechnol 33(2): 187-197.
Vasale, J., F. Boyar, et al. (2015). “Molecular combing compared to Southern blot for measuring D4Z4 contractions in FSHD.” Neuromuscul Disord 25(12): 945-951.
Vasileva, E. A., O. U. Shuvalov, et al. (2015). “Genome-editing tools for stem cell biology.” Cell Death Dis 6: e1831.
Veres, A., B. S. Gosis, et al. (2014). “Low incidence of off-target mutations in individual CRISPR-Cas9 and TALEN targeted human stem cell clones detected by whole-genome sequencing.” Cell Stem Cell 15(1): 27-30.
Villarreal, D. D., K. Lee, et al. (2012). “Microhomology directs diverse DNA break repair pathways and chromosomal translocations.” PLoS Genet 8(11): e1003026.
Vogelstein, B. and K. W. Kinzler (1999). “Digital PCR.” Proc Natl Acad Sci USA 96(16): 9236-9241.
Vouillot, L., A. Thelie, et al. (2015). “Comparison of T7E1 and surveyor mismatch cleavage assays to detect mutations triggered by engineered nucleases.” G3 (Bethesda) 5(3): 407-415.
Wagner, R., P. Debbie, et al. (1995). “Mutation detection using immobilized mismatch binding protein (MutS).” Nucleic Acids Res 23(19): 3944-3948.
Wang, X., Y. Wang, et al. (2015). “Unbiased detection of off-target cleavage by CRISPR-Cas9 and TALENs using integrase-defective lentiviral vectors.” Nat Biotechnol 33(2): 175-178.
White, M. K., W. Hu, et al. (2015). “The CRISPR/Cas9 genome editing methodology as a weapon against human viruses.” Discov Med 19(105): 255-262.
Wu, X., D. A. Scott, et al. (2014). “Genome-wide binding of the CRISPR endonuclease Cas9 in mammalian cells.” Nat Biotechnol 32(7): 670-676.
Yamano, T., H. Nishimasu, et al. (2016). “Crystal Structure of Cpf1 in Complex with Guide RNA and Target DNA.” Cell 165(4): 949-962.
Yang, L., M. Guell, et al. (2013). “Optimization of scarless human stem cell genome editing.” Nucleic Acids Res 41(19): 9049-9061.
Zetsche, B., J. S. Gootenberg, et al. (2015). “Cpf1 is a single RNA-guided endonuclease of a class 2 CRISPR-Cas system.” Cell 163(3): 759-771.
Zhang, Y., N. Heidrich, et al. (2013). “Processing-independent CRISPR RNAs limit natural transformation in Neisseria meningitidis.” Mol Cell 50(4): 488-503.
Zhu, X., Y. Xu, et al. (2014). “An efficient genotyping method for genome-modified animals and human cells generated with CRISPR/Cas9 system.” Sci Rep 4: 6420.

1. A method for detecting, characterizing, quantifying, or
determining the efficiency of, a gene or genome editing procedure or event comprising:
editing a target nucleic acid(s) in a gene or genome and detecting or quantifying at least one genetic modification, deletion, duplication, amplification, translocation, insertion or inversion in the edited target nucleic acid using molecular combing.

2. The method of claim 1, wherein the editing comprises non-homologous end-joining (NHEJ) in a double strand break in the target nucleic acid(s).

3. The method of claim 1, wherein the editing comprises homologous recombination in the target nucleic acid(s) comprising at least one of allelic homologous recombination, gene conversion, non-allelic homologous recombination (NAHR), break-induced replication (BIR), or single strand annealing (SSA).

4. The method of claim 1, wherein the editing procedure comprises activating endogenous cellular repair machinery and contacting the target nucleic acid with a zinc finger nuclease.

5. The method of claim 1, wherein the editing comprises activation of endogenous cellular repair machinery and contacting the target nucleic acid(s) with at least one TALEN (Transcription activator-like effector nuclease).

6. The method of claim 1, wherein the editing comprises activating endogenous cellular repair machinery and contacting the target nucleic acid(s) with at least one meganuclease.

7. The method of claim 1, wherein the editing comprises activating endogenous cellular repair machinery and contacting the target nucleic acid(s) with at least one meganuclease of the LAGLIDADG (SEQ. ID NO: 1) family.

8. The method of claim 1, wherein the editing comprises activating endogenous cellular repair machinery and contacting the target nucleic acid(s) with at least one I-CreI or I-SceI meganuclease.

9. The method of claim 1, wherein the editing comprises activating endogenous cellular repair machinery and contacting the target nucleic acid(s) with a CRISPR/Cas9 system or CRISPR/Cas9 variant system.

10. The method of claim 1,
wherein the editing comprises activating endogenous cellular repair machinery and contacting the target nucleic acid(s) with a type I CRISPR/Cas9 system;
wherein the editing comprises activating endogenous cellular repair machinery and contacting the target nucleic acid(s) with a type II CRISPR/Cas9 system;
wherein the editing comprises activating endogenous cellular repair machinery and contacting the target nucleic acid(s) with a type III CRISPR/Cas9 system;
wherein the editing comprises activation of endogenous cellular repair machinery and contact of target nucleic acid(s) with a type IV CRISPR/Cas9 system;
wherein the editing comprises activating endogenous cellular repair machinery and contacting the target nucleic acid(s) with a type V CRISPR/Cas9 system; or
wherein the editing comprises activating endogenous cellular repair machinery and contacting the target nucleic acid(s) with a type VI CRISPR/Cas9 system.

11. The method of claim 1, wherein the editing produces a nucleic acid rearrangement that knocks out a gene.

12. The method of claim 1,
wherein the editing produces a nucleic acid rearrangement that mutates the target nucleic acid(s);
wherein the editing produces a nucleic acid rearrangement comprising a gene correction;
wherein the editing produces a nucleic acid rearrangement comprising a deletion;
wherein the editing produces a nucleic acid rearrangement comprising an insertion;
wherein the editing produces a nucleic acid rearrangement comprising a duplication;
wherein the editing produces a nucleic acid rearrangement comprising an amplification;
wherein the editing produces a nucleic acid rearrangement comprising a translocation; or
wherein the editing produces a nucleic acid rearrangement comprising an inversion.

13. The method of claim 1 that quantifies a number of nucleic acid rearrangements produced by the editing of the target nucleic acid(s).

14. The method of claim 1 that quantifies a number of nucleic acid rearrangements produced by the editing of the target nucleic acid(s) faster or with a higher degree of accuracy than a conventional quantification method selected from the group consisting of restriction site selection, PAGE-based genotyping assay, enzymatic mismatch cleavage-based assay, subcloning a target region, high-resolution melting curve (HRM) analysis, Next-Gen gene sequencing, and droplet digital PCR.

15. The method of claim 1, wherein the genome or gene editing procedure or event occurs in vivo or in a sample obtained from in vivo, optionally after treatment of a subject by gene therapy or with a polynucleotide, drug, radiation, immunological agent or other therapy.

16. The method according to claim 1, wherein said editing comprises:
contacting the target nucleic acid that has been edited with an engineered nuclease or meganuclease(s), with an unedited control target sequence, and
comparing said edited target nucleic acid sequence with the sequence of the unedited control target sequence.

17. The method according to claim 1, wherein a number of deletions or other unwanted or unexpected genetic events in the target nucleic acid(s) as well as a number of desired or expected edits to the target nucleic acid(s) are quantified by molecular combing.

18. The method of claim 17, wherein the editing is performed using an engineered nuclease or meganuclease.

19. The method according to claim 1, wherein said target nucleic acid(s) comprise BRCA1 genomic DNA.

20. A method for determining the efficiency, accuracy or specificity of a polynucleotide editing procedure that uses at least one modified nuclease comprising:
(i) editing one or more polynucleotide(s) of interest using at least one modified nuclease,
(ii) contacting the edited polynucleotide(s) with labelled polynucleotide(s) that hybridize to them and performing molecular combing of the fluorescent labeled polynucleotides, and
(iii) comparing the edited polynucleotides hybridized to said labelled polynucleotides to one or more control polynucleotides, which have not been treated with the modified nuclease, hybridized to said labelled polynucleotide(s), thus determining the efficiency, accuracy or specificity of the polynucleotide editing procedure using the modified nuclease; and
(iv) optionally, selecting a modified nuclease based polynucleotide editing procedure that is most accurate or efficient for correction or modification of a particular polynucleotide of interest.

21. The method according to claim 1, wherein the target nucleic acid(s) or a target polynucleotide of interest comprises BRCA1 genomic DNA.


SEQUENCE LISTING (50 sequences)

SEQ ID NO: 1 - protein, 9 aa, Artificial Sequence
  Amino acid sequence motif
LAGLIDADG

SEQ ID NO: 2 - DNA, 20 bp, Artificial Sequence
  Primer  Sce-Ia_For
gaattccagtccgtccgata

SEQ ID NO: 3 - DNA, 20 bp, Artificial Sequence
  Primer Sce-1_Rev
cgacgggatctatcatcgtt

SEQ ID NO: 4 - DNA, 20 bp, Artificial Sequence
  Primer Sce-1b_For
tccgtccgatattaccctgt

SEQ ID NO: 5 - DNA, 20 bp, Artificial Sequence
  Primer  Sce-1_Rev
cgacgggatctatcatcgtt

SEQ ID NO: 6 - DNA, 20 bp, Artificial Sequence
  Primer  Sce-2_For
gctcggatccactagtccag

SEQ ID NO: 7 - DNA, 20 bp, Artificial Sequence
  Primer  Sce-2_Rev
gtgctgcaaggcgattaagt

SEQ ID NO: 8 - DNA, 20 bp, Artificial Sequence
  Primer  Sce-3_For
caccaaaatcaacgggactt

SEQ ID NO: 9 - DNA, 20 bp, Artificial Sequence
  Primer  Sce-3_Rev
agccagtaagcagtgggttc

SEQ ID NO: 10 - DNA, 20 bp, Artificial Sequence
  BRCA-Left-gRNA1
ggggtgcggtttattcatac

SEQ ID NO: 11 - DNA, 20 bp, Artificial Sequence
  BRCA-Left_gRNA4
cctgaggcgggtggatcatg

SEQ ID NO: 12 - DNA, 20 bp, Artificial Sequence
  BRCA-Left-gRNA7
attcatacaggtagtgagag

SEQ ID NO: 13 - DNA, 20 bp, Artificial Sequence
  BRCA-Right-gRNA4
ccacaccaccaattaccaca

SEQ ID NO: 14 - DNA, 20 bp, Artificial Sequence
  BRCVA-Right-gRNA9
atgggagaaggtcatagatg

SEQ ID NO: 15 - DNA, 20 bp, Artificial Sequence
  BRCA-Right-gRNA12
gtggaggcagagattacaca

SEQ ID NO: 16 - DNA, 20 bp, Artificial Sequence
  BRCA-Left-PCR-F
tggcttcaaagagactgcga

SEQ ID NO: 17 - DNA, 20 bp, Artificial Sequence
  BRCA-Left-PCR-R
tgtcagcatttggctccact

SEQ ID NO: 18 - DNA, 20 bp, Artificial Sequence
  BRCA-Left-PCR-F
tggcttcaaagagactgcga

SEQ ID NO: 19 - DNA, 24 bp, Artificial Sequence
  BRCA-Right-PCR-R
ggccagtgtagctggagtaatttg

SEQ ID NO: 20 - DNA, 26 bp, Artificial Sequence
  Primer  Synt1b_For
tttagaaaatacatcaccccagttcc

SEQ ID NO: 21 - DNA, 27 bp, Artificial Sequence
  Primer  Synt1b_Rev
ttgaaataccaccttttcatttccaga

SEQ ID NO: 22 - DNA, 20 bp, Artificial Sequence
  Primer  S7b_For
ggaggcagaaattgggcata

SEQ ID NO: 23 - DNA, 21 bp, Artificial Sequence
  Primer S7b_Rev
ttctgacccacagactctcca

SEQ ID NO: 24 - DNA, 25 bp, Artificial Sequence
  Primer  S11_For
ctcgattcaaaaacaaaatgtggcc

SEQ ID NO: 25 - DNA, 20 bp, Artificial Sequence
  Primer  S11_Rev
atgccgtagttggtccaacg

SEQ ID NO: 26 - DNA, 22 bp, Artificial Sequence
  Primer  S12_For
aaaaactctacatcaggggaca

SEQ ID NO: 27 - DNA, 27 bp, Artificial Sequence
  Primer S12_Rev
aaagaaagaaaaagtaaaaactaaagg

SEQ ID NO: 28 - DNA, 23 bp, Artificial Sequence
  crRNA sequence(5′ -> 3′)
  misc_feature (21)..(21): n is a, c, g, or t
attcatacaggtagtgagagnrg

SEQ ID NO: 29 - DNA, 23 bp, Artificial Sequence
  DNA target sequence sequence (5′ -> 3′)
aatcatacaggtagtgacagaag

SEQ ID NO: 30 - DNA, 23 bp, Artificial Sequence
  DNA target sequence sequence (5′ -> 3′)
attcagacaggtagagagaggag

SEQ ID NO: 31 - DNA, 23 bp, Artificial Sequence
  DNA target sequence sequence (5′ -> 3′)
attcatacaggtactgtgagaag

SEQ ID NO: 32 - DNA, 23 bp, Artificial Sequence
  crRNA sequence(5′ -> 3′)
  misc_feature (21)..(21): n is a, c, g, or t
ccacaccaccaattaccacanrg

SEQ ID NO: 33 - DNA, 23 bp, Artificial Sequence
  DNA target sequence sequence (5′ -> 3′)
ccacaccaccaattaccacaagg

SEQ ID NO: 34 - DNA, 23 bp, Artificial Sequence
  crRNA sequence(5′ -> 3′)
  misc_feature (21)..(21): n is a, c, g, or t
attcatacaggtagtgagagnrg

SEQ ID NO: 35 - DNA, 23 bp, Artificial Sequence
  AaTCATACAGGTAGTGAcAGAAG
aatcatacaggtagtgacagaag

SEQ ID NO: 36 - DNA, 23 bp, Artificial Sequence
  DNA target sequence sequence (5′ -> 3′)
attcagacaggtagagagaggag

SEQ ID NO: 37 - DNA, 23 bp, Artificial Sequence
  DNA target sequence sequence (5′ -> 3′)
attcatacaggtactgtgagaag

SEQ ID NO: 38 - DNA, 23 bp, Artificial Sequence
  crRNA sequence(5′ -> 3′)
  misc_feature (21)..(21): n is a, c, g, or t
atgggagaaggtcatagatgnrg

SEQ ID NO: 39 - DNA, 23 bp, Artificial Sequence
  DNA target sequence sequence (5′ -> 3′)
atggaagaaggtaatagatgagg

SEQ ID NO: 40 - DNA, 23 bp, Artificial Sequence
  crRNA sequence(5′ -> 3′)
  misc_feature (21)..(21): n is a, c, g, or t
attcatacaggtagtgagagnrg

SEQ ID NO: 41 - DNA, 23 bp, Artificial Sequence
  DNA target sequence sequence (5′ -> 3′)
aatcatacaggtagtgacagaag

SEQ ID NO: 42 - DNA, 23 bp, Artificial Sequence
  DNA target sequence sequence (5′ -> 3′)
attcagacaggtagagagaggag

SEQ ID NO: 43 - DNA, 23 bp, Artificial Sequence
  DNA target sequence sequence (5′ -> 3′)
attcatacaggtactgtgagaag

SEQ ID NO: 44 - DNA, 23 bp, Artificial Sequence
  crRNA sequence(5′ -> 3′)
  misc_feature (21)..(21): n is a, c, g, or t
gtggaggcagagattacacanrg

SEQ ID NO: 45 - DNA, 23 bp, Artificial Sequence
  DNA target sequence sequence (5′ -> 3′)
gtggaggcagaggctacacatgg

SEQ ID NO: 46 - DNA, 23 bp, Artificial Sequence
  DNA target sequence sequence (5′ -> 3′)
gtgaaggcagaggttacacaggg

SEQ ID NO: 47 - DNA, 23 bp, Artificial Sequence
  DNA target sequence sequence (5′ -> 3′)
gttgaggcagtgattacacatgg

SEQ ID NO: 48 - DNA, 23 bp, Artificial Sequence
  DNA target sequence sequence (5′ -> 3′)
gagtaggcagagattacacaggg

SEQ ID NO: 49 - DNA, 23 bp, Artificial Sequence
  DNA target sequence sequence (5′ -> 3′)
atggagtcagagattacacaaag

SEQ ID NO: 50 - DNA, 23 bp, Artificial Sequence
  DNA target sequence sequence (5′ -> 3′)
gtggaggcagagattagagaggg